(19) United States
(12) Patent Application Publication
(10) Pub. No.: US 2005/0142610 A1
(43) Pub. Date: Jun. 30, 2005

(54) Variant polypeptides containing plekstrin homology domains and uses therefor

(75) Inventors: David G. Lambright, Marlborough, MA (US); Michael P. Czech, Westborough, MA (US); Thomas Cronin, Worcester, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 10/962,128

(22) Filed: Oct. 7, 2004

Related U.S. Application Data
(60) Provisional application No. 60/509,777, filed on Oct. 7, 2003.

(51) Int. Cl. G01N 33/53; C07H 21/04; C07K 14/705

(52) U.S. Cl. 435/7.1; 530/350; 536/23.5

(57) ABSTRACT

The instant invention provides polypeptides comprising variant pleckstrin homology (PH) domains. The invention provides polypeptides having increased or decreased binding specificity for a phosphatidylinositide molecule to which the PH domain naturally binds. Further, the invention provides polypeptides having increased binding specificity for a phosphatidylinositide molecule to which the PH domain naturally does not bind.

### RELATED APPLICATIONS

This application claims priority to U.S. Provisional Application No. 60/509777, filed Oct. 7, 2003, the entire contents of which are incorporated herein by reference.

### GOVERNMENT FUNDING

The work described herein was supported, at least in part, by funding from the National Institute of Health Grant DK 60564.

### BACKGROUND

Lipid binding domains that target intracellular membranes play a crucial role in the assembly of signaling and trafficking complexes and in membrane remodeling events such as vesicle budding, phagocytosis, and cell motility. The biological significance of membrane targeting is underscored by the prevalence of lipid binding domains, which rank amongst the most common domains in the eukaryotic proteome, and by the discovery of major proto-oncogene proteins and tumor suppressors containing essential lipid binding domains and/or lipid metabolic activities that regulate membrane association (1-4), There are several major classes of lipid binding domains including pleckstrin homology (PH), FYVE (acronym of Fabl, YOTB, Vacl, and EEA1), plant homeodomain (PHD), phox homology (PX), and C2 (named for homology with protein kinase C, PKC) domains as well as variety of smaller domain families and peptide motifs. The variation in physical properties and recognition mechanisms between and within families is striking.

Pleckstrin homology domains are commonly found in eukaryotic signaling proteins. The family possesses multiple functions including the ability to bind inositol phosphates. PH domains have been found to possess inserted domains, e.g., such as syntorphins in PLC gamma, and to be inserted within other domains. Mutations in Burtons tyrosine kinase within its PH domain causes X-linked agammaglobulinaemia (XLA) in patients.

Multiple species of 3′-phosphorylated inositol lipids are thought to be involved in a number of cellular signaling and membrane trafficking pathways, including membrane ruffling (Parker, P. J.(1994) Curr. Biol. 5:577; Wennstrom, S. et al. (1994) Curr. Biol. 4:385), chemotaxis (Parker, P. J.(1994) Curr. Biol. 5:577; Wennstrom, S. et al. (1994) Curr. Biol. 4:385), secretory responses (Parker, P. J.(1994) Curr. Biol. 5:577; Wennstrom, S. et al. (1994) Curr. Biol. 4:385), membrane trafficking of growth factor receptors (Okada, T. et al. (1994) J. Biol. Chem. 269:3568; Kanai, F. et al. (1993) Biochem. Biophys. Res. Commun. 195:762), insulin secretion, cell regulated adhesion and insulin-mediated translocation of glucose transporters to the cell surface (reviewed in Czech (1995) Annu. Rev. Nutri. 15:441-471). A relatively large, constitutive pool of PI 3-phosphate is present in resting cells, while very low levels of PI 3,4-biphosphate and PI 3,4,5-triphosphate are rapidly increased in response to a number of external cellular stimuli (reviewed in Cantley et al. (1991) Cell 64:281-302 and Kapeller, R. and L. C. Cantley (1994) Bioessays 16:565-578). The pool of PI 3-phosphate may be largely due to PI (Bonnema, J. D. et al. (1994) J. Exp. Med. 180:1427; Yano, H. et al. (1993) J. Biol. Chem. 268:25846)-kinases such as PtdIns 3-kinase (Bonnema, J. D. et al. (1994) J. Exp. Med. 180:1427; Yano, H. et al. (1993) J. Biol. Chem. 268:25846), a mammalian homolog of the yeast VPS34 protein (Herman, P. K. and S. D. Emir (1990) Mol. Cell. Biol. 10:6742-6754), which can utilize only PI as substrate. In contrast, a second category of PI 3-kinases, isoforms of the p110 PI 3-kinase, are capable of phosphorylating PI 4-phosphate and PI 4,5-bisphosphate at the 3′ position (Hiles et al. (1992) Cell 70:419-429; Hu et al. (1993) Mol. Cell. Biol. 13:7677-7688; Kippel et al. (1994) Mol. Cell. Biol. 14:2676-2685; Stoyanov et al. (1995) Science 269:690-693). These enzymes apparently contribute to the regulated pools of PI 3,4-P₂ and PI-3,4,5-P₅ stimulated by receptor or non-receptor tyrosine kinase activation (in the case of isoforms p110 and p110β) or G protein activation (in the case of p110γ). The existence of multiple PI 3-kinase isoforms suggests the influence of multiple signaling pathways on these enzymes and, possibly, divergent reactions of the individual 3′-phosphoinositides.

The extensive literature on phosphoinositide metabolism by lipid kinases and phosphatases is covered in two recent reviews (5, 6). Given a high negative charge density, distributed over 2-4 phosphates in close proximity, it is not surprising that a strong positive electrostatic potential should be a common feature of the various domains that recognize phosphoinositides. What is more remarkable, in view of the pseudo-symmetry of the D-myo-inositol head group, is the high degree of stereochemical selectivity that lipid binding domains have evolved to distinguish even the most structurally similar phosphoinositides.

Several groups have reported a novel protein module of approximately 100 amino acids termed the pleckstrin homology (PH) domain located at the carboxy-terminal of several proteins involved in signal transduction processes (Haslam et al. (1993) Nature 363:309-310; Mayer et al. (1993) Cell 73:629-630; Musacchio et al. (1993) Trends Biochem. Sci. 18:343-348). PH domains have been implicated in the binding to membranes containing PI 4,5-bisphosphate, as well as to the binding of several proteins βγ subunits (Gβγ) of heterotrimeric G proteins (Touhara et al. (1994) J. Biol. Chem. 269:10217-10220; Satoshi et al. (1994) Proc. Natl. Acad. Sci. USA 91:11256-11260; Lemmon et al. (1995) Proc. Natl. Acad. Sci. USA 92:10472-10476), protein kinase C (Yao et al. (2994) Proc. Natl. Acad. Sci. USA 91:9175-9179), WD motifs (Wang et al. (1994) Biochem. Biophys. Res. Commun. 203:29-35

PH domains have been found in a number of proteins including protein kinase C α, phospholipase C-δ1, the serine/threonine kinase known variously as protein kinase B, Akt and Rac (Burgering, B. M. T. and P. J. Coffer (1995) Nature 376:599-602; Franke et al. (1995) Cell 81:727-736; Coffer, P. J. and J. R. Woodgett (1991) Eur. J Biochem. 201:475-481) among others.

Phosphoinositides are second messengers that have been shown to play a critical role in cell survival, membrane trafficking, insulin regulation, adhesion, migration and cytoskeletal dynamics. Based on the prevalence of PH domains and the biological importance of phosphoinositide, a need exisists for understanding and controlling the selectively of various PH domain containing polypeptides for one or more given phosphoinositides.

### SUMMARY OF THE INVENTION

The instant invention is based on the discovery that mutants of PH domains result in polypeptide that have significantly altered affinity (i.e., increased or decreased) for given phosphoinositides. Variants that differ by as little as one amino acid in the PH domain can have completely different ligand recognition and/or can have vastly different affinity for the natural ligand when compared to the wild type polypeptide.

Accordingly, in at least one embodiment, the invention provides polypeptides comprising a variant PH domain. In one specific embodiment, the polypeptide has increased binding specificity for a phosphatidylinositide molecule to which the PH domain naturally binds. Alternatively, the polypeptide has decreased binding specificity for a phosphatidylinositide molecule to which the PH domain naturally binds.

In another related embodiment, the polypeptide has increased binding specificity for a phosphatidylinositide molecule to which the PH domain naturally does not bind. In yet another related embodiment, the polypeptide has decreased binding specificity for a phosphatidylinositide molecule to which the PH domain naturally does not bind. In specific embodiments, the phosphatidylinositide molecule is phosphatidylinositol-3,4,5 (PI-3,4,5)P3 or phosphatidylinositol-4,5 (PI-4,5)P2.

In related embodiments, the variant PH domains have increased or decreased affinity for phosphatidylinositol-3,4,5 (PI-3,4,5)P3 or phosphatidylinositol-4,5 (PI-4,5)P2.

In one specific embodiment the variant PH domain has at least one, two, or three glycine residues inserted in the β1/β2 loop as compared to the wild-type sequence.

In another embodiment, the variant PH domain comprises an amino acid substitution in a residue that does not contact the head group of a given phosphatidylinositol.

In another specific embodiment, the PH domain is present within a Grp1/ARNO/ Cytohesin family polypeptide.

In another embodiment, the invention provides a method of using a PH domain variant to selectively detect the presence of a specific phosphatidylinositide. In related embodiments, the phosphatidylinositide molecule is phosphatidylinositol-3,4,5 (PI-3,4,5)P3 or phosphatidylinositol-4,5 (PI-4,5)P2.

In specific embodiments of the invention, the polypeptide comprising a variant PH domain has a 10, 100, or 1000 fold higher specificity for a given phosphatidylinositide molecule than the wild-type polypeptide.

In one specific embodiment, the polypeptide comprising a variant PH domain has lost the ability to bind and/or recognize the natural ligand (e.g., phosphatidylinositol-3,4,5 (PI-3,4,5)P3 or phosphatidylinositol-4,5 (PI-4,5)P2).

In another embodiment, the invention provides a polypeptide comprising a variant PH domain wherein the variant (i) increases the affinity of the PH domain for one ligand while not changing the affinity for a second ligand: (ii) increases the affinity of the PH domain for one ligand while decreasing the affinity for a second ligand; or (iii) increases the affinity of the PH domain for one ligand while increasing the affinity for a second ligand. In certain embodiments the second ligand is a natural ligand of the PH domain. In another embodiment, the second ligand is not a natural ligand of the PH domain.

In another embodiment, the invention provides a polypeptide comprising a variant PH domain wherein the variant (i) decreases the affinity of the PH domain for one ligand while not changing the affinity for a second ligand; (ii) decreases the affinity of the PH domain for one ligand while decreasing the affinity for a second ligand; or (iii) variant decreases the affinity of the PH domain for one ligand while increasing the affinity for a second ligand. In certain embodiments the second ligand is a natural ligand of the PH domain. In another embodiment, the second ligand is not a natural ligand of the PH domain.

In one embodiment the invention provides a variant GRP1 polyeptide with a substitution selected from the group consisting of K273A, K282A, R284A, Y295F, R277A, R277C, V278A, V278C, K279A, K279C, T280A, T280C, R305A, K343A, N354A, and H355A of SEQ ID NO:1. In a related embodiment the invention provides a variant GRP1 polyeptide having one or more of the following substitutions: of K273A, K282A, R284A, Y295F, R277A, R277G, V278A, V278C, K279A, K279G, T280A, T280G, R305A, K343A, N354A, and/or H355A of SEQ ID NO:1.

In one embodiment the invention provides a variant ARNO polyeptide with a substitution selected from the group consisting of K273A, K283A, R285A, Y296F, R278G, V279G, K280G, T281G, R306A, K344A, N355A, and H356A of SEQ ID NO:3. In a related embodiment the invention provides a variant ARNO polyeptide having one or more of the following substitutions: K273A, K283A, R285A, Y296F, R278G, V279G, K280G, T281G, R306A, K344A, N355A, and/or H356A of SEQ ID NO:3.

### BRIEF DESCRIPTION OF THE DRAWINGS

FIG. 1 Depicts PI 3-Kinase Signaling

In response to extracellular signals, PI 3-kinases catalyze the formation of 3-phosphoinositides. These second messengers are critical to cellular functions such as cell survival, membrane trafficking, insulin regulation, adhesion, migration and cytoskeletal dynamics.

FIG. 2 Depicts PH Domain Structures

Space filling models of PH domains bound to the phosphoinositides IP3 and IP4. Grp1 Btk and Dapp1 all bind IP4 in a similar orientation but do so making contacts with different loops. PLCδ binds IP3 in a flipped orientation compared to the way Grp1, Btk and Dapp1 bind IP4. Grp1 possesses a hairpin insertion of the β6/δ7 loop that Btk and Dapp1 are missing. Btk and Dapp1 possess longer β1/β2 loops than Grp1. Differences in the loop regions may explain the wide range of ligand affinities.

FIG. 3 Depicts the Phosphoinositides Relevant to PH Domains

The major signaling phosphoinositides are represented as red and yellow stick models. Pleckstrin homology domains may bind to a diverse selection of phosphoinositides with varying degrees of specificity.

FIG. 4 Depicts the Comparison of β1/β2 Loops of PH Domains

These PH domains recognize phosphoinositides with a wide range of affinities and specificities. Despite almost 90% identity between the Grp1, ARNO and Cytohesin domains, there are drastic differences in their affinity and specificity for PIP3 and PIP2. The affinity for PIP2 over PIP3 can be affected by presence or absence of a third glycine in the β1/β2 loop.

FIG. 5 Depicts the Structure of the ARNO PH Domain Bound to IP3

A. A 1.8 Å resolution xray data set was collected on crystals of ARNO bound to IP3. In the first round of refinement, electron density for the IP3 head group is present.

B. A ribbon diagram depicts the structure of ARNO bound to IP3.

C. The PH domains of ARNO and Grp1 bound to IP3 and IP4 respectively is shown for comparison purposes. Many of the residues in Grp1 that contact the 3 and 4 phosphates of IP4 contact the 4 phosphate of IP3 in ARNO. Consequently, many of the residues that contact the 4 and 5 phosphate of IP4 in Grp1 make contact with the 5 phosphate of IP3 in ARNO.

FIG. 6 Depicts the Selection Against IP3 in Grp1

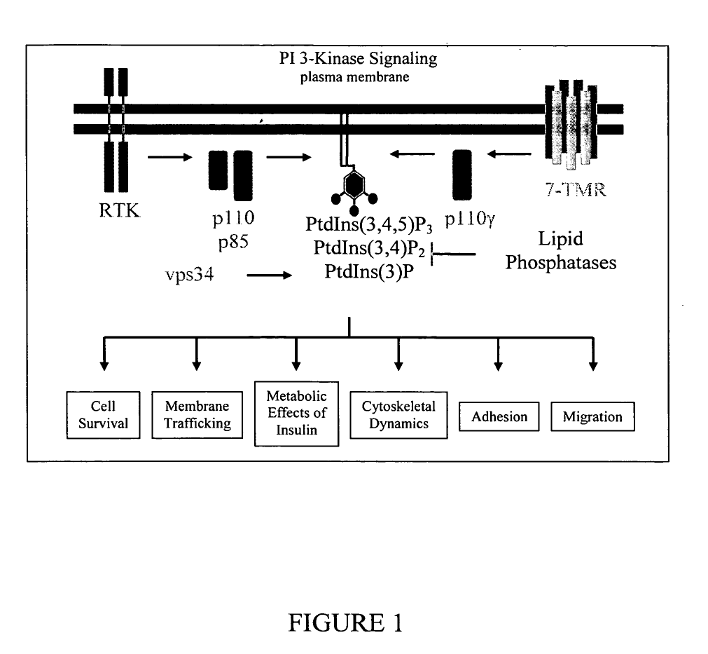

A. The β1/β2 loops of ARNO bound to IP3 and Grp1 bound to IP4 have been superimposed. The IP4 in the Grp1 structure has been removed to show the placement of the IP3 in relation to Grp1. The shorter β1/β2 loop of Grp1 (2G) brings a valine in close proximity to the 1 phosphate of IP3. The longer β1/β2 loop in ARNO keeps the valine at 279 away from the 1 phosphate in IP3.

B. The Grp1 PH domain bound to IP4 is shown with the IP3 of ARNO modeled in the phosphoinositide binding pocket. ARNO binds IP3 in a shifted or rotated manner compared to the way in which Grp1 and Btk bind IP4. The contacts made with the 3 phosphate in Grp1 are made with the 4 phosphate in ARNO, while those residues that contact the 4 and 5 phosphates in Grp make contact with the 5 phosphate in ARNO. Furthermore, ARNO does not bind IP3 like PLCδ, which binds the ligand in a flipped orientation compared to Grp1 and Btk. This suggests that ARNO binds IP3 in a somewhat novel manner.

C. The β1/β2 loops of ARNO bound to IP3 and Grp1 bound to IP4 are superimposed with the IP4 of Grp1 removed. IP3 and valine are rendered to show proximity of atomic radii. The valine of Grp1 clashes sterically with IP3 while the valine of ARNO is juxtaposed away from IP3. With its longer β1/β2 loop, ARNO can accommodate IP3 and IP4. Grp1 possesses a shorter β1/β2 loop that cannot accommodate IP3 as easily as IP4. This may explain the specificity Grp1 has for IP4 over IP3.

FIG. 7 Depicts the Structures of the ARNO and Grp1 PH Domains Bound to IP4

A. A ribbon diagram is shown of a 2.3 Å resolution crystal structure of the ARNO PH domain bound to IP4. Note the canonical beta barrel and variability loops that surround the opening of the barrel.

B. The ARNO and Grp1 structures bound to IP4 are superimposed. IP4 is bound in the same orientation and mode in Grp1 and ARNO. Note the shorter β1 /β2 loop of Grp1 is brought close to the 1 phosphate of IP4 while the longer β1/β2 loop of ARNO is farther away from IP4. The longer β1/β2 loop of ARNO can accommodate binding IP3 and IP4 with little specificity while the shorter loop of Grp1 helps enforce binding IP4 over IP3.

FIG. 8 depicts the Electron Density for Unbound Grp1 (3G) PH domain A sigma weighted map of the electron density for the phosphoinositide binding pocket of the Grp1 (3G) PH domain is shown. Despite the lack of an IP3 ligand, there are sulfate ions in the IP3 position, suggesting a preset position for recognizing phosphoinositides in a limited orientation.

FIG. 9 depicts an Isothermal Titration Calorimetry Experiments

Isothermal titration calorimetry (ITC) experiments were performed on wild type and mutant constructs of GST tagged Grp1 (2G) and Arno (3G). IP3 or IP4, was titrated into a sample cell containing appropriate Grp1 family construct.

A. Sample curve of IP4 being injected into a cell containing wild type ARNO. Each peak represents heat released upon IP4 binding.

B. Plotting integrated heats of binding for wild type ARNO and IP4 follow a single site binding model.

FIG. 10 Depicts the Results of ITC Experiments

A. The structure of the Grp1 PH domain is depicted in ribbon form with the specificity determining regions color coded. Contacts between the side chains and IP4 are represented as dotted lines.

B. Constructs of GST tagged Grp1(2G) were titrated with IP4. The dissociation constant (Kd) for each interaction was determined and compared to the wild type. The relative Kds are plotted for each Grp1 mutant.

C. & D. Constructs of GST tagged ARNO (3G) were titrated with IP3 or IP4. The dissociation constant (Kd) for each interaction was determined and compared to the wild type. The relative Kds are plotted for each ARNO mutant. The V279G mutant bound IP3 and IP4 with higher affinity than wild type.

FIG. 11 Depicts an Alignment of Various Pleckstrin Homology Domains Against a Consensus PH Domain.

FIGS. 12A and B depict the PH domains of GRP1 (SEQ ID NO:5) and ARNO (SEQ ID NO:6), respectively.

### DETAILED DESCRIPTION

The instant invention is based on the discovery that mutants of PH domains result in polypeptide that have significantly altered affinity (i.e., increased or decreased) for given phosphoinositides. Variants that differ by as little as one amino acid in the PH domain can have completely different ligand recognition and/or can have vastly different affinity for the natural ligand when compared to the wild type polypeptide.

Accordingly, in at least one embodiment, the invention provides polypeptides comprising a variant PH domain. In one specific embodiment, the polypeptide has increased binding specificity for a phosphatidylinositide molecule to which the PH domain naturally binds. Alternatively, the polypeptide has decreased binding specificity for a phosphatidylinositide molecule to which the PH domain naturally binds.

In another related embodiment, the polypeptide has increased binding specificity for a phosphatidylinositide molecule to which the PH domain naturally does not bind. In yet another related embodiment, the polypeptide has decreased binding specificity for a phosphatidylinositide molecule to which the PH domain naturally does not bind. In specific embodiments, the phosphatidylinositide molecule is phosphatidylinositol-3,4,5 (PI-3,4,5)P3 or phosphatidylinositol-4,5 (PI-4,5)P2.

In related embodiments, the variant PH domains have increased or decreased affinity for phosphatidylinositol-3,4,5 (PI-3,4,5)P3 or phosphatidylinositol-4,5 (PI-4,5)P2.

In one specific embodiment the variant PH domain has at least one, two, or three glycine residues inserted in the β1/β2 loop as compared to the wild-type sequence.

In another embodiment, the variant PH domain comprises an amino acid substitution in a residue that does not contact the head group of a given phosphatidylinositol.

In another specific embodiment, the PH domain is present within a Grp1/ARNO/ Cytohesin family polypeptide.

In another embodiment, the invention provides a method of using a PH domain variant to selectively detect the presence of a specific phosphatidylinositide. In related embodiments, the phosphatidylinositide molecule is phosphatidylinositol-3,4,5 (PI-3,4,5)P3 or phosphatidylinositol-4,5 (PI-4,5)P2.

In specific embodiments of the invention, the polypeptide comprising a variant PH domain has a 10, 100, or 1000 fold higher specificity for a given phosphatidylinositide molecule than the wild-type polypeptide.

In one specific embodiment, the polypeptide comprising a variant PH domain has lost the ability to bind and/or recognize the natural ligand (e.g., phosphatidylinositol-3,4,5 (PI-3,4,5)P3 or phosphatidylinositol-4,5 (PI-4,5)P2).

In another embodiment, the invention provides a polypeptide comprising a variant PH domain wherein the variant (i) increases the affinity of the PH domain for one ligand while not changing the affinity for a second ligand: (ii) increases the affinity of the PH domain for one ligand while decreasing the affinity for a second ligand; or (iii) increases the affinity of the PH domain for one ligand while increasing the affinity for a second ligand. In certain embodiments the second ligand is a natural ligand of the PH domain. In another embodiment, the second ligand is not a natural ligand of the PH domain.

In another embodiment, the invention provides a polypeptide comprising a variant PH domain wherein the variant (i) decreases the affinity of the PH domain for one ligand while not changing the affinity for a second ligand; (ii) decreases the affinity of the PH domain for one ligand while decreasing the affinity for a second ligand; or (iii) variant decreases the affinity of the PH domain for one ligand while increasing the affinity for a second ligand. In certain embodiments the second ligand is a natural ligand of the PH domain. In another embodiment, the second ligand is not a natural ligand of the PH domain.

In one embodiment the invention provides a variant GRP1 polyeptide with a substitution selected from the group consisting of K273A, K282A, R284A, Y295F, R277A, R277C, V278A, V278C, K279A, K279C, T280A, T280C, R305A, K343A, N354A, and H355A of SEQ ID NO:1(Table 1).

TABLE 1
GrP1 polypeptide sequence

| | |
|---|---|
| LOCUS | NP_004218 399 aa linear PRI |
| DEFINITION | pleckstrin homology, Sec7 and coiled/coil domains 3; cytohesin 3; |
| | ARF nucleotide-binding site opener 3; general receptor of |
| | phosphoinositides 1 [Homo sapiens]. |
| ACCESSION | NP_004218 |
| VERSION | NP_004218.1 GI: 4758968 |
| DBSOURCE | REFSEQ: accession NM_004227.3 |
| KEYWORDS | . |
| SOURCE | Homo sapiens (human) |
| ORGANISM | Homo sapiens |
| | Eukaryota; Metazoa; Chordata; Craniata; Vertebrate; Euteleostomi; |
| | Mammalia; Eutheria; Primates; Catarrhini; Hominidae; Homo. |
| REFERENCE | 1 (residues 1 to 399) |
| AUTHORS | Ogasawara, M., Kim, S. C., Adamik, R., Togawa, A., Ferrans, V. J., |
| | Takeda, K., Kirby, M., Moss, J. and Vaughan, M. |
| TITLE | Similarities in function and gene structure of cytohesin-4 and |
| | cytohesin-1, guanine nucleotide-exchange proteins for |
| | ADP-ribosylation factors |
| JOURNAL | J. Biol. Chem. 275 (5), 3221-3230 (2000) |
| MEDLINE | 20119275 |
| PUBMED | 10652308 |
| REFERENCE | 2 (residues 1 to 399) |
| AUTHORS | Venkateswarlu, K., Gunn-Moore, F., Oatey, P. B., Tavare, J. M. and |
| | Cullen, P. J. |
| TITLE | Nerve growth factor- and epidermal growth factor-stimulated |
| | translocation of the ADP-ribosylation factor-exchange factor GRP1 |
| | to the plasma membrane of PC12 cells requires activation of |
| | phosphatidylinositol 3-kinase and the GRP1 pleckstrin homology |
| | domain |
| JOURNAL | Biochem. J. 335 (Pt 1), 139-146 (1998) |
| MEDLINE | 98416124 |
| PUBMED | 9742223 |
| REFERENCE | 3 |
| AUTHORS | Franco, M., Boretto, J., Robineau, S., Monier, S., Goud, B., Chardin, P. |
| | and Chavrier, P. |
| TITLE | ARNO3, a Sec7-domain guanine nucleotide exchange factor for ADP |
| | ribosylation factor 1, is involved in the control of Golgi |
| | structure and function |
| JOURNAL | Proc. Natl. Acad. Sci. U.S.A. 95 (17), 9926-9931 (1998) |
| MEDLINE | 98374282 |
| PUBMED | 9707577 |
| REFERENCE | 4 (residues 1 to 399) |
| AUTHORS | Klarlund, J. K., Guilherme, A., Holik, J. J., Virbasius, J. V., Chawla, A. |
| | and Czech, M. P. |
| TITLE | Signaling by phosphoinositide-3,4,5-trisphosphate through proteins |
| | containing pleckstrin and Sec7 homology domains |
| JOURNAL | Science 275 (5308), 1927-1930 (1997) |
| MEDLINE | 97228176 |
| PUBMED | 9072969 |
| COMMENT | REVIEWED REFSEQ: This record has been curated by NCBI staff. The |
| | reference sequence was derived from CB988199.1, AJ223957.1, |
| | BC028717.1 and BG620295.1. |
| | Summary: This gene encodes a member of the PSCD (pleckstrin |
| | homology, Sec7 and coiled-coil domains) family. PSCD family members |
| | have identical structural organization that consists of an |
| | N-terminal coiled-coil motif, a central Sec7 domain, and a |
| | C-terminal pleckstrin homology (PH) domain. The coiled-coil motif |
| | is involved in homodimerization, the Sec7 domain contains |
| | guanine-nucleotide exchange protein (GEP) activity, and the PH |
| | domain interacts with phospholipids and is responsible for |
| | association of PSCDs with membranes. Members of this family appear |
| | to mediate the regulation of protein sorting and membrane |
| | trafficking. This encoded protein is involved in the control of |
| | Golgi structure and function, and it may have a physiological role |
| | in regulating ADP-ribosylation factor protein 6 (ARF) functions, in |
| | addition to acting on ARF1. |
| FEATURES | Location/Qualifiers |
| source | 1..399 |
| | /organism=“Homo sapiens” |
| | /db_xref=“taxon:9606” |
| | /chromosome=“7” |
| | /map=“7p22.2” |
| Protein | 1..399 |
| | /product=“pleckstrin homology, Sec7 and coiled/coil |
| | domains 3” |
| | /note=“cytohesin 3; ARF nucleotide-binding site opener 3; |
| | general receptor of phosphoinositides 1” |
| Region | 65..248 |
| | /region_name=“Sec7 domain. The Sec7 domain is a |
| | guanine-nucleotide-exchange-factor (GEF) for the pfam00025 |
| | family” |
| | /note=“Sec7” |
| | /db_xref=“CDD:pfam01369” |
| Region | 66..248 |
| | /region_name=“Sec7 domain” |
| | /note=“Sec7” |
| | /db_xref=“CDD:14836” |
| Region | 265..381 |
| | /region_name=“Pleckstrin homology domain” |
| | /note=“PH” |
| | /db_xref=“CDD:24224” |
| Region | 265..381 |
| | /region_name=“Pleckstrin homology domain. Domain commonly |
| | found in eukaryotic signalling proteins. The domain family |
| | possesses multiple functions including the abilities to |
| | bind inositol phosphates, and various proteins. PH domains |
| | have been found to possess inserted domains (such as in |
| | PLC gamma, syntrophins) and to be inserted within other |
| | domains. Mutations in Brutons tyrosine kinase (Btk) within |
| | its PH domain cause X-linked agammaglobulinaemia (XLA) in |
| | patients. Point mutations cluster into the positively |
| | charged end of the molecule around the predicted binding |
| | site for phosphatidylinositol lipids” |
| | /note=“PH” |
| | /db_xref=“CDD:smart00233” |
| CDS | 1..399 |
| | /gene=“PSCD3” |
| | /coded_by=“NM_004227.3:105..1304” |
| | /note=“go_component: membrane fraction [goid 0005624] |
| | [evidence E] [pmid 9742223]; |
| | go_component: plasma membrane [goid 0005886] [evidence E] |
| | [pmid 9742223]; |
| | go_function: phosphatidylinositol binding [goid 0005545] |
| | [evidence E] [pmid 9742223]; |
| | go_function: ARF guanyl-nucleotide exchange factor |
| | activity [goid 0005086] [evidence E] [pmid 9707577]; |
| | go_function: inositol-1,4,5-triphosphate receptor activity |
| | [goid 0008095] [evidence P] [pmid 9742223]; |
| | go_process: vesicle-mediated transport [goid 0016192] |
| | [evidence E] [pmid 9707577]” |
| | /db_xref=“GeneID:9265” |
| | /db_xref=“LocusID:9265” |
| | /db_xref=“MIM:605081” |

| | | | | | | |
|---|---|---|---|---|---|---|
| 1 | mdedgggegg | gvpedlslee | reelldirrr | kkeliddier | lkyeiaevmt | eidnltsvee |
| 61 | skttqrnkqi | amgrkkfnmd | pkkgiqflie | ndllqssped | vaqflykgeg | lnktvigdyl |
| 121 | gerdefnikv | lqafvelhef | adlnlvqalr | qflwsfrlpg | eaqkidrmme | afasryclcn |
| 181 | pgvfqstdtc | yvlsfaiiml | ntslhnhnvr | dkptaerfia | mnrgineggd | lpeellrnly |
| 241 | esiknepfki | peddgndlth | tffnpdregw | llklggrvkt | wkrrwfiltd | nclyyfeytt |
| 301 | dkeprgiipl | enlsireved | prkpncfely | npshkgqvik | ackteadgrv | vegnhvvyri |
| 361 | sapspeekee | wmksikasis | rdpfydmlat | rkrriankk | | |

In a related embodiment the invention provides a variant GRP 1 polyeptide having one or more of the following substitutions: of K273A, K282A, R284A, Y295F, R277A, R277G, V278A, V278C, K279A, K279G, T280A, T280G, R305A, K343A, N354A, and/or H355A of SEQ ID NO:1.

In one embodiment the invention provides a variant ARNO polyeptide with a substitution selected from the group consisting of K273A, K283A, R285A, Y296F, R278G, V279G, K280G, T281G, R306A, K344A, N355A, and H356A of SEQ ID NO:3 (Table II).

TABLE II
ARNO polypeptide sequence

| | |
|---|---|
| LOCUS | NP_004219 399 aa linear |
| DEFINITION | pleckstrin homology, Sec7 and coiled/coil domains 2 isoform 2; |
| | pleckstrin homology, Sec7 and coiled/coil domains 2; cytohesin 2 |
| | [Homo sapiens]. |
| ACCESSION | NP_004219 |
| VERSION | NP_004219.1 GI: 4758966 |
| DBSOURCE | REFSEQ: accession NM_004228.3 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (residues 1 to 399) |
| AUTHORS | Huh, M., Han, J. H., Lim, C. S., Lee, S. H., Kim, S., Kim, E. and |
| | Kaang, B. K. |
| TITLE | Regulation of neuritogenesis and synaptic transmission by msec7-1, |
| | a guanine nucleotide exchange factor, in cultured Aplysia neurons |
| JOURNAL | J. Neurochem. 85 (1), 282-285 (2003) |
| MEDLINE | 22529431 |
| PUBMED | 12641750 |
| REMARK | GeneRIF: The overexpression of ARNO, another mammalian GEF, |
| | produces extensive neuritogenesis in Aplysia neurons |
| REFERENCE | 2 (residues 1 to 399) |
| AUTHORS | Smith, J. S., Tachibana, I., Pohl, U., Lee, H. K., Thanarajasingam, U., |
| | Portier, B. P., Ueki, K., Ramaswamy, S., Billings, S. J., |
| | Mohrenweiser, H. W., Louis, D. N. and Jenkins, R. B. |
| TITLE | A transcript map of the chromosome 19q-arm glioma tumor suppressor |
| | region |
| JOURNAL | Genomics 64 (1), 44-50 (2000) |
| MEDLINE | 20175430 |
| PUBMED | 10708517 |
| REFERENCE | 3 (residues 1 to 399) |
| AUTHORS | Ogasawara, M., Kim, S. C., Adamik, R., Togawa, A., Ferrans, V. J., |
| | Takeda, K., Kirby, M., Moss, J. and Vaughan, M. |
| TITLE | Similarities in function and gene structure of cytohesin-4 and |
| | cytohesin-1, guanine nucleotide-exchange proteins for |
| | ADP-ribosylation factors |
| JOURNAL | J. Biol. Chem. 275 (5), 3221-3230 (2000) |
| MEDLINE | 20119275 |
| PUBMED | 10652308 |
| REFERENCE | 4 (residues 1 to 399) |
| AUTHORS | Venkateswarlu, K., Oatey, P. B., Tavare, J. M. and Cullen, P. J. |
| TITLE | Insulin-dependent translocation of ARNO to the plasma membrane of |
| | adipocytes requires phosphatidylinositol 3-kinase |
| JOURNAL | Curr. Biol. 8 (8), 463-466 (1998) |
| MEDLINE | 98217355 |
| PUBMED | 9550703 |
| REFERENCE | 5 (residues 1 to 399) |
| AUTHORS | Cherfils, J., Menetrey, J., Mathieu, M., Le Bras, G., Robineau, S., |
| | Beraud-Dufour, S., Antonny, B. and Chardin, P. |
| TITLE | Structure of the Sec7 domain of the Arf exchange factor ARNO |
| JOURNAL | Nature 392 (6671), 101-105 (1998) |
| MEDLINE | 98169075 |
| PUBMED | 9510256 |
| REFERENCE | 6 (residues 1 to 399) |
| AUTHORS | Mossessova, E., Gulbis, J. M. and Goldberg, J. |
| TITLE | Structure of the guanine nucleotide exchange factor Sec7 domain of |
| | human arno and analysis of the interaction with ARF GTPase |
| JOURNAL | Cell 92 (3), 415-423 (1998) |
| MEDLINE | 98135767 |
| PUBMED | 9476900 |
| REFERENCE | 7 (residues 1 to 399) |
| AUTHORS | Frank, S., Upender, S., Hansen, S. H. and Casanova, J. E. |
| TITLE | ARNO is a guanine nucleotide exchange factor for ADP-ribosylation |
| | factor 6 |
| JOURNAL | J. Biol. Chem. 273 (1), 23-27 (1998) |
| MEDLINE | 98079021 |
| PUBMED | 9417041 |
| REFERENCE | 8 (residues 1 to 399) |
| AUTHORS | Chardin, P., Paris, S., Antonny, B., Robineau, S., Beraud-Dufour, S., |
| | Jackson, C. L. and Chabre, M. |
| TITLE | A human exchange factor for ARF contains Sec7- and |
| | pleckstrin-homology domains |
| JOURNAL | Nature 384 (6608), 481-484 (1996) |
| MEDLINE | 97100951 |
| PUBMED | 8945478 |
| REFERENCE | 9 (residues 1 to 399) |
| AUTHORS | Kolanus, W., Nagel, W., Schiller, B., Zeitlmann, L., Godar, S., |
| | Stockinger, H. and Seed, B. |
| TITLE | Alpha L beta 2 integrin/LFA-1 binding to ICAM-1 induced by |
| | cytohesin-1, a cytoplasmic regulatory molecule |
| JOURNAL | Cell 86 (2), 233-242 (1996) |
| MEDLINE | 96319726 |
| PUBMED | 8706128 |
| COMMENT | REVIEWED REFSEQ: This record has been curated by NCBI staff. The |
| | reference sequence was derived from X99753.1 and U70728.1. |
| | Summary: Pleckstrin homology, Sec7 and coiled/coil domains 2 |
| | (PSCD2) is a member of the PSCD family. Members of this family have |
| | identical structural organization that consists of an N-terminal |
| | coiled-coil motif, a central Sec7 domain, and a C-terminal |
| | pleckstrin homology (PH) domain. The coiled-coil motif is involved |
| | in homodimerization, the Sec7 domain contains guanine-nucleotide |
| | exchange protein (GEP) activity, and the PH domain interacts with |
| | phospholipids and is responsible for association of PSCDs with |
| | membranes. Members of this family appear to mediate the regulation |
| | of protein sorting and membrane trafficking. PSCD2 exhibits GEP |
| | activity in vitro with ARF1, ARF3, and ARF6. PSCD2 protein is 83% |
| | homologous to PSCD1. |
| | Transcript Variant: This transcript (2) is missing 3 bp in the PH |
| | domain region, which results in a protein isoform missing a single |
| | glycine residue. |
| FEATURES | Location/Qualifiers |
| source | 1..399 |
| | /organism=“Homo sapiens” |
| | /db_xref=“taxon:9606” |
| | /chromosome=“19” |
| | /map=“19q13.3” |
| Protein | 1..399 |
| | /product=“pleckstrin homology, Sec7 and coiled/coil |
| | domains 2 isoform 2” |
| | /note=“pleckstrin homology, Sec7 and coiled/coil domains |
| | 2; cytohesin 2” |
| Region | 13..54 |
| | /region_name=“Coiled-coil domain” |
| Region | 60..243 |
| | /region_name=“Sec7 domain. The Sec7 domain is a |
| | guanine-nucleotide-exchange-factor (GEF) for the |
| | pfam00025 |
| | family” |
| | /note=“Sec7” |
| | /db_xref=“CDD:pfam01369” |
| Region | 61..243 |
| | /region_name=“Sec7 domain” |
| | /note=“Sec7” |
| | /db_xref=“CDD:14836” |
| Region | 72..252 |
| | /region_name=“Sec7 domain” |
| Region | 260..375 |
| | /region_name=“Pleckstrin homology domain” |
| | /note=“PH” |
| | /db_xref=“CDD:24224” |
| Region | 260..375 |
| | /region_name=“Pleckstrin homology domain. Domain commonly |
| | found in eukaryotic signalling proteins. The domain |
| | family |
| | possesses multiple functions including the abilities to |
| | bind inositol phosphates, and various proteins. PH |
| | domains |
| | have been found to possess inserted domains (such as in |
| | PLC gamma, syntrophins) and to be inserted within other |
| | domains. Mutations in Brutons tyrosine kinase (Btk) within |
| | its PH domain cause X-linked agammaglobulinaemia (XLA) in |
| | patients. Point mutations cluster into the positively |
| | charged end of the molecule around the predicted binding |
| | site for phosphatidylinositol lipids” |
| | /note=“PH” |
| | /db_xref=“CDD:smart00233” |
| Region | 262..375 |
| | /region name=“PH domain” |
| CDS | 1..399 |
| | /gene=“PSCD2” |
| | /coded_by=“NM_004228.3:159..1358” |
| | /note=“go_component: kinesin complex [goid 0005871] |
| | [evidence IEA]; |
| | go_component: membrane fraction [goid 0005624] [evidence |
| | TAS] [pmid 9417041]; |
| | go_component: plasma membrane [goid 0005886] [evidence |
| | TAS] [pmid 9417041]; |
| | go_function: ARF guanyl-nucleotide exchange factor |
| | activity [goid 0005086] [evidence TAS] [pmid 9417041]; |
| | go_function: guanyl-nucleotide release factor activity |
| | [goid 0019839] [evidence IEA]; |
| | go_process: actin cytoskeleton reorganization [goid |
| | 0007012] [evidence TAS] [pmid 9417041]; |
| | go_process: endocytosis [goid 0006897] [evidence TAS] |
| | [pmid 9417041]” |
| | /db_xref=“GeneID:9266” |
| | /db_xref=“LocusID:9266” |
| | /db_xref=“MIM:602488” |

| | | | | | | |
|---|---|---|---|---|---|---|
| 1 | medgvyeppd | ltpeermele | nirrrkqell | veiqrlreel | seamsevegl | eanegsktlq |
| 61 | rnrkmamgrk | kfnmdpkkgi | qflvenellq | ntpeeiarfl | ykgeglnkta | igdylgeree |
| 121 | lnlavlhafv | dlheftdlnl | vqalrqflws | frlpgeaqki | drmmeafaqr | yclcnpgvfq |
| 181 | stdtcyvlsf | avimlntslh | npnvrdkpgl | erfvamnrgi | neggdlpeel | lrnlydsirn |
| 241 | epfkipeddg | ndlthtffnp | dregwllklg | grvktwkrrw | filtdnclyy | feyttdkepr |
| 301 | giiplenlsi | revddprkpn | cfelyipnnk | gqlikackte | adgrvvegnh | mvyrisaptq |
| 361 | eekdewiksi | qaavsvdpfy | emlaarkkri | svkkkqeqp | | |

In a related embodiment the invention provides a variant ARNO polyeptide having one or more of the following substitutions: K273A, K283A, R285A, Y296F, R278G, V279G, K280G, T281G, R306A, K344A, N355A, and/or H356A of SEQ ID NO:3.

PH Domains

Lipid binding domains that target intracellular membranes play a crucial role in the assembly of signaling and trafficking complexes and in membrane remodeling events such as vesicle budding, phagocytosis, and cell motility. The biological significance of membrane targeting is underscored by the prevalence of lipid binding domains, which rank amongst the most common domains in the eukaryotic proteome, and by the discovery of major proto-oncogene proteins and tumor suppressors containing essential lipid binding domains and/or lipid metabolic activities that regulate membrane association (1-4), There are several major classes of lipid binding domains including pleckstrin homology (PH), FYVE (acronym of Fabl, YOTB, Vacl, and EEA1), plant homeodomain (PHD), phox homology (PX), and C2 (named for homology with protein kinase C, PKC) domains as well as variety of smaller domain families and peptide motifs. The variation in physical properties and recognition mechanisms between and within families is striking.

The “pleckstrin homology” (PH) domain is a domain of about 100 residues that is present in a wide range of proteins involved in intracellular signaling or as constituents of the cytoskeleton. The pleckstrin homology domain is given PFAM accession number PF00169.

Lipids, Head Groups, and Phosphoinositides

Membranes consisting of phospholipid bilayers represent a ubiquitous component of all cells. With a large repertoire of chemically distinct lipids, the composition of biological membranes is highly complex and variable, depending both on the type of cell and organelle of interest. Lipid composition also varies within organelles, giving rise to microdomains with distinct physiochemical properties that reflect both the stereochemical and electrostatic characteristics of the head group as well as the length, saturation, and branching of the hydrocarbon chains. The more abundant phospholipids include phosphatidyl choline (PC), phosphatidyl ethanolamine (PE) and phosphatidyl serine (PS). PC and PE are neutral zwitterions, whereas PS bears a net negative charge. Though less abundant, mono and polyphosphorlyted derivatives of phosphatidyl inositol (PtdIns), collectively referred to as ‘phosphoinositides’, play a disproportionately critical role as targets for the known lipid binding domains. Several physiochemical properties that distinguish inositol from other common lipid head groups may well have contributed to the convergent evolution of functionally related yet structurally distinct phosphoinositide binding domains. With five equatorial hydroxyl substituents and a single axial hydroxyl group at the D2 position, the semi-rigid cyclohexane based ring of D-myo inositol represents a prominent landmark against the backdrop of typically smaller head groups. Reversible phosphorylation of the D3, D4, and/or D5 hydroxyl groups transforms an otherwise weakly anionic phospholipid, with a net negative charge of −1, into seven distinct derivatives: three monophosphates, three bisphosphates, and a single trisphosphate, with high net negative charges of −3, −5, and −7, respectively. The extensive literature on phosphoinositide metabolism by lipid kinases and phosphatases is covered in two recent reviews (5, 6). Given a high negative charge density, distributed over 2-4 phosphates in close proximity, it is not surprising that a strong positive electrostatic potential should be a common feature of the various domains that recognize phosphoinositides. What is more remarkable, in view of the pseudo-symmetry of the D-myo-inositol head group, is the high degree of stereochemical selectivity that lipid binding domains have evolved to distinguish even the most structurally similar phosphoinositides.

PH Domains

PH domains comprise one of the largest and most intensively investigated families of lipid binding domains (7-9). They were initially identified in signaling, cytoskeletal and metabolic proteins as evolutionarily conserved modules of −120 amino acids with weak homology to pleckstrin, a protein kinase C (PKC) substrate in platelets (10-13). As estimated by the human genome project, there are over 250 proteins containing one or more PH domains, making them one of the most common domains (14, 15). Of the PH domains that have been characterized, the majority bind weakly to phosphoinositides with little or no selectivity. An elite subset representing 10-20% of PH domains exhibit relatively high affinity (Kd for the head group in the low uM to nM range) and varying degrees of specificity for polyphosphoinositides (16-19).

Despite high sequence variability, NMR and crystal structures of more than dozen different PH domains have established a canonical core fold consisting of a seven stranded partly open p barrel, capped at one end by a C-terminal a helix (7, 20-28). Outside the core regions, the loops connecting the various secondary structural elements are best characterized as hypervariable with respect to composition, length, and structure, although similarities are apparent within sub-families. As discussed below, the hypervariable loops play a critical role in determining the functional properties, in particular the diverse affinity and specificity for phosphoinositides. Nevertheless, one property characteristic of PH domains that bind phosphosphoinositides, with either high or low affinity/selectivity, is a strongly dipolar electrostatic potential, with the positive lobe typically centered near the open end of the central p barrel (29, 30). This bulk electrostatic property accounts, at least in part, for the weak phosphoinositide affinities and specificities of many PH domains that correlate directly with the net charge of the head group (18). In these cases, preferences for phosphoinositides over other acidic lipids presumably derive from the higher negative charge density of mono- and polyphosphoinositides rather than stereochemical determinants.

A significant number of PH domains have be shown to bind polyphosphoinositides with relatively high affinity and with specificities dependent on the arrangement of phosphate groups attached to the inositol ring. These include the PtdIns(4,5)P2 specific PLC8 PH domain as well as the PtdIns(3,4,5)P3 specific PH domains of Bruton's Tyrosine Kinase (Btk) and General Receptor for 3-Phosphoinositides (Grp1) (16-19, 31-33). The PH domains of Dual Adapter for Phosphotyrosine and 3-Phosphoinositides (Dapp1) and the protein kinase B proto-oncogene (PKB/Akt) are promiscuous for Ptdms(3,4,)P₂ and PtdIns(3,4,5)P3 yet discriminate against PtdIns(4,5)P₂ (1, 17-19, 34-36). In a peculiar evolutionary twist, splice variants within the Grp1 family of PH domains, in which a single glycine residue is inserted at the N-terminus of the p1/p2 loop, bind promiscuously to either PtdIns(4,5)P₂ or PtdIns(3,4,5)P₂ (37, 38). Several of these PH domains have the property of binding with higher affinity to the head group than to the corresponding lipid (18). At least for the PH domains of Grp1, Btk, Dapp1, PKB/Akt, and PLC8, which target the plasma membrane, the affinity for the head group appears to be sufficient to drive membrane association, although other interactions may influence the precise localization within membrane microdomains.

Crystal structures of the aforementioned PH domains in complex with inositol polyphosphates provide insight into the determinants of phosphoinositide recognition (22, 23, 28, 39, 40). These PH domains conserve a basic signature motif, K—Xₘ—(R/K)—X—R—Xₙ—(Y/N), with the first lysine located near the C-terminus of the (β 1 strand, the (R/K)—X—R sequence near the N-terminus of the β 2 stand, and a tyrosine residue in the β3 strand (17-19, 22, 23}. In a variation on theme, the PKB/Akt PH domain substitutes the signature tyrosine with a functionally analogous asparagine residue from the β 3/β 4 loop (28). The first and third basic residues of the signature motif line the most deeply buried and positively charged region of the binding site and, together with the signature tyrosine/asparigine residue, mediate stereochemically equivalent interactions with either the 3- and 4-phosphates (Grp1, Btk, Dapp1, and PKB/Akt) or the 4-and 5-phosphates (PLCδ). Mutational analyses indicate that the signature residues, in particular the first and third basic residues, are critical but not sufficient for head group binding (17, 19, 31, 32, 41). With the exception of the PLCδ PH domain, the majority of the interactions with the head group are mediated by basic and polar residues from three ‘specificity determining regions’ (SDKs) corresponding to the hypervariable β 1/β 2, β 3/β 4, and β 6/β 7 loops, which flank the phospoinositide binding site at open end of the β barrel. Main chain NH groups in β 1/β2 loop mediate interactions with either the 5-phosphate (Btk and Grp1) or the 1-phosphate (PKB/Akt), reminiscent of P-loop interactions with phosphate groups in nucleotide binding proteins.

An important lesson from these studies is that similar specificities can be achieved through quite distinct structural mechanisms. For example, the relatively long (11 residue) P1/P2 loop in the Btk PH domain accounts for all of the interactions with the 5-phosphate and half of the contacts with the 4-phosphate. In the Grp1 PH domain, a twenty residue insertion in the P6/P7 loop adopts a P hairpin structure, which straddles the 4- and 5-phosphates, thereby compensating for a short (6 residue) p1/p2 loop. Equally significant structural variations are observed between the Dapp1 and PKB/Akt PH domains.

Grp1 Family PH Domains

The highly homologous proteins Grp1, ARNO (Arf nucleotide binding site opener), and cytohesin define a functionally related family with a modular domain architecture consisting of an N-terminal heptad repeat, a domain with exchange activity for Arf GTPases, a PH domain, and a C-terminal polybasic sequence. Alternative splice variants of ARNO and cytohesin give rise to full length proteins that differ only in the number glycine residues at the N-terminus of the β 1/β 2 loop in the PH domain. The ‘diglycine’ (2G) variants exhibit a strong selectivity for PtdIns(3,4,5)P₃, with a 30 fold higher affinity compared with that for PtdIns(4,5)P2- In contrast, the ‘triglycine’ (3G) variants, which contain a glycine insertion relative to the diglycine variants, bind both PtdIns(4,5)P2 and PtdIns(3,4,5)P3 with comparable affinity.

Polypeptides

An “isolated” or “purified” protein or biologically active portion thereof is substantially free of cellular material or other contaminating proteins from the cell or tissue source from which the variant PH domain containing protein is derived, or substantially free from chemical precursors or other chemicals when chemically synthesized. The language “substantially free of cellular material” includes preparations of variant PH domain containing protein in which the protein is separated from cellular components of the cells from which it is isolated or recombinantly produced. In one embodiment, the language “substantially free of cellular material” includes preparations of variant PH domain containing protein having less than about 30% (by dry weight) of non-variant PH domain containing protein (also referred to herein as a “contaminating protein”), more preferably less than about 20% of non-variant PH domain containing protein, still more preferably less than about 10% of non-variant PH domain containing protein, and most preferably less than about 5% non-variant PH domain containing protein. When the variant PH domain containing protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The term “GAC family proteins” in intended to include polypeptides that are homologous to the GRP1/ARNO/Cytohesion polypeptides. Further, this term is intended to include polypeptides that are homologous to the PH domain from the GAC family of proteins, or that contain a PH domain that is homologous to a PH domain from a GAC family polypeptide.

The term “PH domain containing protein” is intended to include polypeptides that naturally have a PH domain or that have been genetically engineered to have a PH domain (e.g., chimeric or fusion proteins). Proteins that naturally have a PH domain include, but are not limited to, Grp1, Btk, Dapp1, PKB/Adk, and PCLδ.

The language “substantially free of chemical precursors or other chemicals” includes preparations of variant PH domain containing protein in which the protein is separated from chemical precursors or other chemicals which are involved in the synthesis of the protein. In one embodiment, the language “substantially free of chemical precursors or other chemicals” includes preparations of variant PH domain containing protein having less than about 30% (by dry weight) of chemical precursors or non- variant PH domain containing chemicals, more preferably less than about 20% chemical precursors or non- variant PH domain containing chemicals, still more preferably less than about 10% chemical precursors or non- variant PH domain containing chemicals, and most preferably less than about 5% chemical precursors or non-variant PH domain containing chemicals.

In a preferred embodiment, the variant PH domain is a variant of the amino acid sequence shown in SEQ ID NO:5 or 6. In other embodiments, the variant PH domain containing protein is substantially identical to SEQ ID NO:5 or 6, and retains the functional activity of a Pleckstrin homology domain, yet differs in amino acid sequence. Accordingly, in another embodiment, the variant PH domain containing protein is a protein which comprises an amino acid sequence at least about 90%, 91%, 92%, 93%, 94%, 95%, 96%, 97%, 98%, 99%, 99.5% or more identical to SEQ ID NO:1 or 3, or the PH domain of SEQ ID NO:1 or 3 (SEQ ID NO:5 or 6). Further, the PH domain variant has 1, 2, 3, 4, 5, 6, 7, 8, 9, 10 or more amino acid insertions, deletions, or substitutions as compared to the wild-type sequence.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available online through the website of the Genetics Computer Group), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online through the website of the Genetics Computer Group), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of Meyers, E. and Miller, W. ((1988) Comput. Appl. Biosci. 4:11-17) which has been incorporated into the ALIGN program (version 2.0 or 2.0U), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to OP nucleic acid molecules of the invention. BLAST protein searches can be performed with the XBLAST program, score=100, wordlength=3 to obtain amino acid sequences homologous to OP protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See the website of the National Center for Biotechnology Information.

Variants of the PH domain containing proteins can be generated by mutagenesis, e.g., discrete point mutation or truncation of a PH domain containing protein. Specific biological effects can be elicited by introducing mutations into the PH domain containing protein (see the Examples).

In one embodiment, a library of PH domain variants is generated by combinatorial mutagenesis at the nucleic acid level and is encoded by a gene library. A library of PH domain variants can be produced by, for example, enzymatically ligating a mixture of synthetic oligonucleotides into gene sequences such that a degenerate set of potential PH domain sequences is expressible as individual polypeptides, or alternatively, as a set of larger fusion proteins (e.g., for phage display) containing the set of PH domain sequences therein. There are a variety of methods which can be used to produce libraries of potential PH domain variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential OP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PH domain containing proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify PH domain variants (Arkin and Youvan (1992) Proc. Natl. Acad. Sci. USA 89:7811-7815; Delagrave et al. (1993) Protein Eng. 6(3):327-331).

Nucleic Acid Molecules

One aspect of the invention pertains to nucleic acid molecules that encode variant PH domain containing proteins. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule of the present invention, e.g., a nucleic acid molecule that is a variant of SEQ ID NO:2 or 4 (Table III and IV, respectively) can be isolated using standard molecular biology techniques and the sequence information provided herein.

TABLE III
GrP1 nucleic acid sequence

| | |
|---|---|
| LOCUS | NM_004227 4482 bp mRNA linear |
| DEFINITION | Homo sapiens pleckstrin homology, Sec7 and coiled-coil domains 3 |
| | (PSCD3), mRNA. |
| ACCESSION | NM_004227 |
| VERSION | NM_004227.3 GI: 33946275 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae; Homo. |
| REFERENCE | 1 (bases 1 to 4482) |
| AUTHORS | Ogasawara, M., Kim, S. C., Adamik, R., Togawa, A., Ferrans, V. J., |
| | Takeda, K., Kirby, M., Moss, J. and Vaughan, M. |
| TITLE | Similarities in function and gene structure of cytohesin-4 and |
| | cytohesin-1, guanine nucleotide-exchange proteins for ADP-ribosylation factors |
| JOURNAL | J. Biol. Chem. 275 (5), 3221-3230 (2000) |
| MEDLINE | 20119275 |
| PUBMED | 10652308 |
| REFERENCE | 2 (bases 1 to 4482) |
| AUTHORS | Venkateswarlu, K., Gunn-Moore, F., Oatey, P. B., Tavare, J. M. and |
| | Cullen, P. J. |
| TITLE | Nerve growth factor- and epidermal growth factor-stimulated |
| | translocation of the ADP-ribosylation factor-exchange factor GRP1 |
| | to the plasma membrane of PC12 cells requires activation of |
| | phosphatidylinositol 3-kinase and the GRP1 pleckstrin homology |
| | domain |
| JOURNAL | Biochem. J. 335 (Pt 1), 139-146 (1998) |
| MEDLINE | 98416124 |
| PUBMED | 9742223 |
| REFERENCE | 3 |
| AUTHORS | Franco, M., Boretto, J., Robineau, S., Monier, S., Goud, B., Chardin, P. |
| | and Chavrier, P. |
| TITLE | ARNO3, a Sec7-domain guanine nucleotide exchange factor for ADP |
| | ribosylation factor 1, is involved in the control of Golgi |
| | structure and function |
| JOURNAL | Proc. Natl. Acad. Sci. U.S.A. 95 (17), 9926-9931 (1998) |
| MEDLINE | 98374282 |
| PUBMED | 9707577 |
| REFERENCE | 4 (bases 1 to 4482) |
| AUTHORS | Klarlund, J. K., Guilherme, A., Holik, J. J., Virbasius, J. V., Chawla, A. |
| | and Czech, M. P. |
| TITLE | Signaling by phosphoinositide-3,4,5-trisphosphate through proteins |
| | containing pleckstrin and Sec7 homology domains |
| JOURNAL | Science 275 (5308), 1927-1930 (1997) |
| MEDLINE | 97228176 |
| PUBMED | 9072969 |
| COMMENT | REVIEWED REFSEQ: This record has been curated by NCBI staff. The |
| | reference sequence was derived from CB988199.1, AJ223957.1, |
| | BC028717.1 and BG620295.1. |
| | On Aug 20, 2003 this sequence version replaced gi: 8670548. |
| | Summary: This gene encodes a member of the PSCD (pleckstrin |
| | homology, Sec7 and coiled-coil domains) family. PSCD family |
| | members |
| | have identical structural organization that consists of an |
| | N-terminal coiled-coil motif, a central Sec7 domain, and a |
| | C-terminal pleckstrin homology (PH) domain. The coiled-coil motif |
| | is involved in homodimerization, the Sec7 domain contains |
| | guanine-nucleotide exchange protein (GEP) activity, and the PH |
| | domain interacts with phospholipids and is responsible for |
| | association of PSCDs with membranes. Members of this family |
| | appear to mediate the regulation of protein sorting and membrane |
| | trafficking. This encoded protein is involved in the control of |
| | Golgi structure and function, and it may have a physiological role |
| | in regulating ADP-ribosylation factor protein 6 (ARF) functions, |
| | in addition to acting on ARF1. |
| | COMPLETENESS: complete on the 3′ end. |
| FEATURES | Location/Qualifiers |
| source | 1..4482 |
| | /organism=“Homo sapiens” |
| | /mol_type=“mRNA” |
| | /db_xref=“taxon:9606” |
| | /chromosome=“7” |
| | /map=“7p22.2” |
| gene | 1..4482 |
| | /gene=“PSCD3” |
| | /note=“synonyms:GRP1, ARNO3” |
| | /db_xref=“GeneID:9265” |
| | /db_xref=“LocusID:9265” |
| | /db_xref=“MIM:605081” |
| CDS | 105..1304 |
| | /gene=“PSCD3” |
| | /note=“cytohesin 3; ARF nucleotide-binding site opener 3; |
| | general receptor of phosphoinositides 1; |
| | go_component: membrane fraction [goid 0005624] [evidence |
| | E] [pmid 9742223]; |
| | go_component: plasma membrane [goid 0005886] [evidence E] |
| | [pmid 9742223]; |
| | go_function: phosphatidylinositol binding [goid 0005545] |
| | [evidence E] [pmid 9742223]; |
| | go_function: ARF guanyl-nucleotide exchange factor |
| | activity [goid 0005086] [evidence E] [pmid 9707577]; |
| | go_function: inositol-1,4,5-triphosphate receptor |
| | activity |
| | [goid 0008095] [evidence P] [pmid 9742223]; |
| | go_process: vesicle-mediated transport [goid 0016192] |
| | [evidence E] [pmid 9707577]” |
| | /codon_start=1 |
| | /product=“pleckstrin homology, Sec7 and coiled/coil |
| | domains 3” |
| | /protein_id=“NP_004218.1” |
| | /db_xref=“GI:4758968” |
| | /db_xref=“GeneID:9265” |
| | /db_xref=“LocusID:9265” |
| | /db_xref=“MIM:605081” |
| | translation=“MDEDGGGEGGGVPEDLSLEEREELLDIRRRKKELIDDIERLKYE |

IAEVMTEIDNLTSVEESKTTQRNKQIAMGRKKFNMDPKKGIQFLIENDLLQSSPEDVA
QFLYKGEGLNKTVIGDYLGERDEFNIKVLQAFVELHEFADLNLVQALRQFLWSFRLPG
EAQKIDRMMEAFASRYCLCNPGVFQSTDTCYVLSFAIIMLNTSLHNHNVRDKPTAERF
IAMNRGINEGGDLPEELLRNLYESIKNEPFKIPEDDGNDLTHTFFNPDREGWLLKLGG
RVKTWKRRWFILTDNCLYYFEYTTDKEPRGIIPLENLSIREVEDPRKPNCFELYNPSH
KGQVIKACKTEADGRVVEGNHVVYRISAPSPEEKEEWMKSIKASISRDPFYDMLATRK

| | |
|---|---|
| | RRIANKK” |
| misc_feature | 297..848 |
| | /gene=“PSCD3” |
| | /note=“Sec7; Region: Sec7 domain. The Sec7 domain is a |
| | guanine-nucleotide-exchange-factor (GEF) for the |
| | pfam00025 |
| | family” |
| | /db_xref=“CDD:pfam01369” |
| misc_feature | 897..1247 |
| | /gene=“PSCD3” |
| | /note=“PH; Region: Pleckstrin homology domain. Domain |
| | commonly found in eukaryotic signalling proteins. The |
| | domain family possesses multiple functions including the |
| | abilities to bind inositol phosphates, and various |
| | proteins. PH domains have been found to possess inserted |
| | domains (such as in PLC gamma, syntrophins) and to be |
| | inserted within other domains. Mutations in Brutons |
| | tyrosine kinase (Btk) within its PH domain cause X-linked |
| | agammaglobulinaemia (XLA) in patients. Point mutations |
| | cluster into the positively charged end of the molecule |
| | around the predicted binding site for |
| | phosphatidylinositol |
| | lipids” |
| | /db_xref=“CDD:smart00233” |
| polyA_signal | 4444..4449 |
| | /gene=“PSCD3” |
| polyA_site | 4469 |
| | /gene=“PSCD3” |
| | /evidence=experimental |

| | | | | | | |
|---|---|---|---|---|---|---|
| 1 | tgaggagccg | cccggtcgcc | tgcgcgctcc | ctccggcggc | gtccccagcc | cgcggcccct |
| 61 | ctgctgccgg | cccccggctc | gccggctgcg | ggagtggcct | caagatggat | gaagacggcg |
| 121 | gcggcgaggg | tggtggcgtg | cctgaagacc | tctcattaga | agagagagaa | gaacttctag |
| 181 | acattcgtcg | aagaaaaaag | gaacttattg | atgacattga | gaggctgaaa | tatgaaattg |
| 241 | cagaggtgat | gacagagatc | gacaatctaa | cttccgtaga | ggagagcaaa | acgactcaga |
| 301 | ggaacaaaca | gatagccatg | ggaagaaaga | aattcaacat | ggatcccaaa | aagggaattc |
| 361 | agtttctaat | agaaaatgac | ctgctacaga | gttccccaga | agacgtcgcc | cagttccttt |
| 421 | ataaaggaga | aggcctaaat | aagaccgtca | ttggggacta | cctgggtgaa | agggatgaat |
| 481 | ttaatattaa | agttcttcaa | gcctttgttg | aactccatga | gtttgctgat | ctcaaccttg |
| 541 | tacaagcctt | aaggcagttc | ttatggagct | tcaggctgcc | cggggaggcg | cagaagattg |
| 601 | atcgcatgat | ggaggctttc | gcttctcgct | actgcctgtg | caaccccggg | gtcttccagt |
| 661 | ccacagacac | gtgctacgtg | ctgtcattcg | ccatcatcat | gctcaacacc | agcctccaca |
| 721 | accacaacgt | gcgtgacaag | cccacggcag | aacggttcat | cgccatgaac | cgcggcatca |
| 781 | acgagggcgg | ggacctccct | gaggagctgc | tgaggaattt | gtatgagagc | attaagaacg |
| 841 | agccatttaa | gatcccggag | gacgacggga | acgacctgac | ccacaccttc | ttcaaccccg |
| 901 | accgcgaggg | ctggctcctg | aagctgggag | ggcgtgtgaa | gacctggaag | cgccggtggt |
| 961 | tcatcctgac | cgataactgc | ctctattact | ttgaatacac | aacagataag | gagcccaggg |
| 1021 | gaatcatccc | gttggaaaac | ctcagcatca | gggaggtgga | ggacccccgg | aaacccaact |
| 1081 | gttttgagct | ctacaatccc | agccacaaag | ggcaggtcat | caaggcctgt | aagactgagg |
| 1141 | ccgacggccg | cgtggtagag | gggaaccatg | tggtgtaccg | gatctcagcc | ccgagcccgg |
| 1201 | aggagaagga | ggagtggatg | aaatccatca | aagccagtat | cagcagagat | cccttctatg |
| 1261 | acatgttggc | aacgaggaaa | cgaaggattg | ccaataaaaa | atagctttcc | tggctaaaag |
| 1321 | acccaggtaa | aagacccaac | cccagcagaa | agacaccgcg | ggcggcccct | ccgcggaagg |
| 1381 | cgtggcaggg | aggcagtcgc | cctgcggtgc | aagctgctgc | tccagagcat | accgtggccc |
| 1441 | aggtggtatc | cccaaggcct | cgtgccgtgg | ctggggtcct | gggaggtggt | cgccctgcag |
| 1501 | tgcaagctgc | tgctccagag | cgtaccgtgg | cccagactga | tcctcgaggc | ctcctgccgt |
| 1561 | ggctggggtc | atggtcggct | gcgcatgtcc | agaagcattt | ccttcctgcg | accatcccgg |
| 1621 | cgcccctagg | gggagaagcc | aggacagcag | cttccgctgt | ctccacagca | gacacgggac |
| 1681 | ggattccaca | gacgggagcc | tcattcgtac | catgccaaac | gcattcactc | ggggcagtat |
| 1741 | taaccgttct | agaaagccac | tgttttatag | caaaacagga | aaggaaaagc | taccagtttt |
| 1801 | ttattcagaa | tttttctcag | atatatagga | ttatagcttt | tatatgcctt | tttatattct |
| 1861 | gaaattataa | caaaagatac | tttctaacag | tagtattttt | agaatggcag | ctataaagtt |
| 1921 | aactcctgga | cacaagtata | tactgtgcac | tgaaaaaata | tccatctaca | cagcacccaa |
| 1981 | ggggagggct | gggggcaccg | gcacgggggc | agcgtgcagc | cctgccctgt | caggctgtca |
| 2041 | gacaagcccc | ggggggcagc | aggtgggctc | gggacgggct | gggggaggga | cggccatggc |
| 2101 | acttgggggc | tccagggtga | ctcccatgag | gcctcccttc | aaccaggctt | tttggcccca |
| 2161 | caaatacttt | aagcaaatca | ttaaaattat | aacagttaat | ggtttggggg | tgtttaggct |
| 2221 | gtaactgcta | actcctagga | aacagccttt | tccctggaca | cagatggtcc | atacgctgag |
| 2281 | ccacgtgaaa | ctgctgatgt | tttgtttaga | tgcacacaca | tggcagcgtt | tcatacaggt |
| 2341 | cagcaggtta | gaccggcttt | tgaccatatt | catcgctatt | taaaacctgt | ggcaaaatga |
| 2401 | acgcttattt | tacagacttt | ctaatttgac | cagatttctt | aatgaataga | cacagaatta |
| 2461 | actaaaaaca | gtctcacccc | atgtagtgcg | ccgtgtcctg | agagaggtgc | cctccctacg |
| 2521 | aggagggaag | aacaggccct | ggggtgcaga | ggcccggcac | gtagagaacc | cagatagacg |
| 2581 | ccggtggtgg | aactggtcaa | actccacgcc | cgcctgggag | gttgtcaggt | tgctgtggat |
| 2641 | gtaaggatag | gaggtgccca | gtgctccgct | caaggaaggc | tggatctggg | ccccacctac |
| 2701 | agagagggct | cagggctgga | ccgggggcat | tgtgtgcttg | ggccgacccg | ggccggtggc |
| 2761 | agacgctgtt | ctctgtcggg | agatttgcgt | ccccaggacc | ctgttacaca | gtgggctgtt |
| 2821 | gggttggtgg | ctggcttttc | ctctatggac | ttcctcttcc | tgccccacct | gcataggcac |
| 2881 | acacaccttg | aatctgcacc | ctctggaggg | catctgtact | cctgtgcaaa | atgcccagtc |
| 2941 | cagagacaaa | acctcagact | ttgtgcacct | aggtttcctt | ctcagcagcg | gagactgttc |
| 3001 | tttgagttgc | cttgaagtgg | aggccgagcg | gctgcgggcc | cttcgcctcc | ctgcggctga |
| 3061 | ccttgatgta | gctttaagtc | acactagact | gcagaggggt | ccgaggccag | aaacccctgt |
| 3121 | cctgcatcag | actttcattc | ccacgttctt | aggctttgtt | actgatacct | caaatcggaa |
| 3181 | gttttagttc | tgagaaaggc | aagtcagcgt | tcttgaaatg | cctgactggt | agatatgcaa |
| 3241 | ctctggcctc | cagtcttcca | tgaaaataaa | tgctgcctgg | acccccaccc | agaccacaca |
| 3301 | ctgacggccg | gctccggcgg | tgcccacccc | tcaggctggc | ccggcaccca | agactggcca |
| 3361 | cagccagctc | tgtcagcatg | ttgtgctcgg | acaagctgtt | tccttcttct | gaccaaccca |
| 3421 | ggtgtgacct | ggggatgcag | agctttctgt | tttgggtgtt | gggagaagca | gcaggaagga |
| 3481 | gtcgccagat | gatcaagctc | cccctttgct | gtcatctgtg | aatgagcttc | gccaggtggt |
| 3541 | gggcacctgg | gagccatgca | gaggctgtgg | tgctgagtta | gactccaggt | actttgtggt |
| 3601 | caaaggaaat | cgcctagctc | caggctgtgt | taggacagta | ttagcatgaa | ggctgtgcga |
| 3661 | ccatcatgcc | tgctgatcct | tgaggcaggc | ctggtccaga | aaactctggg | tcagtgactg |
| 3721 | cgcagggcca | gccgctacca | ggacggccct | gaaacaggac | acatctgttt | tttgtccctc |
| 3781 | accctgggca | ggccgcgtca | caatcacagt | cctcctcctc | cccaccctga | cgtctgagcg |
| 3841 | cagggcttga | attgttagtc | ccaactctgg | ccaaagatac | ttttttccag | agacagaggc |
| 3901 | caggaggcag | tgaggggagc | cccgcgggga | ggcggcggcg | actgccacag | cccttccagc |
| 3961 | ctgtcttgct | ggccgccctg | gttcatattt | gagtttaatt | gtactgaccc | tggacccaga |
| 4021 | taagcagcaa | ctttgtgtct | ttggggtcac | agaacatttt | ggggcagttt | aatgtggtac |
| 4081 | caaactgaaa | ataggagcta | tttatagatg | gagcagcact | tagtgcttca | tagaaagcaa |
| 4141 | tgcctatttt | taaagttaca | aacgcagata | tctacataga | tatgctttgc | tgagaagtta |
| 4201 | ggtctgtggt | agaccagaaa | ccacaaattg | actttttttc | ttagaaaata | tttctatttg |
| 4261 | cggtaaatat | agtaatatgt | aaataatgta | catctgttga | tttctggagt | gtctgttatt |
| 4321 | caatgatgta | tatactccca | cagctcgcat | gaaggaacag | cctctattga | tacttggttg |
| 4381 | taaagtgaag | taagattgga | gggtggatgg | ctgtcagagc | tcttgcagat | actgtgttca |
| 4441 | ctaaataaaa | atcacatgta | ttgttaaaaa | aaaaaaaaaa | aa | |

TABLE IIII
ARNO Nucleic Acid sequence

| | |
|---|---|
| LOCUS | NM_004228 1358 bp mRNA linear |
| DEFINITION | Homo sapiens pleckstrin homology, Sec7 and coiled-coil domains 2 |
| | (cytohesin-2) (PSCD2), transcript variant 2, mRNA. |
| ACCESSION | NM_004228 |
| VERSION | NM_004228.3 GI: 10880123 |
| KEYWORDS | . |
| SOURCE | Homo sapiens (human) |
| ORGANISM | Homo sapiens |
| | Eukaryota; Metazoa; Chordata; Craniata; Vertebrata; Euteleostomi; |
| | Mammalia; Eutheria; Primates; Catarrhini; Hominidae, Homo. |
| REFERENCE | 1 (bases 1 to 1358) |
| AUTHORS | Huh, M., Han, J. H., Lim, C. S., Lee, S. H., Kim, S., Kim, E. and Kaang, B. K. |
| TITLE | Regulation of neuritogenesis and synaptic transmission by msec7-1. |
| | a guanine nucleotide exchange factor, in cultured Aplysia neurons |
| JOURNAL | J. Neurochem. 85 (1), 282-285 (2003) |
| MEDLINE | 22529431 |
| PUBMED | 12641750 |
| REMARK | GeneRIF: The overexpression of ARNO, another mammalian GEF, |
| | produces extensive neuritogenesis in Aplysia neurons |
| REFERENCE | 2 (bases 1 to 1358) |
| AUTHORS | Smith, J. S., Tachibana, I., Pohl, U., Lee, H. K., Thanarajasingam, U., |
| | Portier, B. P., Ueki, K., Ramaswamy, S. , Billings, S. J., |
| | Mohrenweiser, H. W., Louis, D. N. and Jenkins, R. B. |
| TITLE | A transcript map of the chromosome 19q-arm glioma tumor suppressor |
| | region |
| JOURNAL | Genomics 64 (1), 44-50 (2000) |
| MEDLINE | 20175430 |
| PUBMED | 10708517 |
| REFERENCE | 3 (bases 1 to 1358) |
| AUTHORS | Ogasawara, M., Kim, S. C., Adamik, R., Togawa, A., Ferrans, V. J., |
| | Takeda, K., Kirby, M., Moss, J. and Vaughan, M. |
| TITLE | Similarities in function and gene structure of cytohesin-4 and |
| | cytohesin-1, guanine nucleotide-exchange proteins for |
| | ADP-ribosylation factors |
| JOURNAL | J. Biol. Chem. 275 (5), 3221-3230 (2000) |
| MEDLINE | 20119275 |
| PUBMED | 10652308 |
| REFERENCE | 4 (bases 1 to 1358) |
| AUTHORS | Venkateswarlu, K., Oatey, P. B., Tavare, J. M. and Cullen, P. J. |
| TITLE | Insulin-dependent translocation of ARNO to the plasma membrane of |
| | adipocytes requires phosphatidylinositol 3-kinase |
| JOURNAL | Curr. Biol. 8 (8), 463-466 (1998) |
| MEDLINE | 98217355 |
| PUBMED | 9550703 |
| REFERENCE | 5 (bases 1 to 1358) |
| AUTHORS | Cherfils, J., Menetrey, J., Mathieu, M., Le Bras, G., Robineau, S., |
| | Beraud-Dufour, S., Antonny, B. and Chardin, P. |
| TITLE | Structure of the Sec7 domain of the Arf exchange factor ARNO |
| JOURNAL | Nature 392 (6671), 101-105 (1998) |
| MEDLINE | 98169075 |
| PUBMED | 9510256 |
| REFERENCE | 6 (bases 1 to 1358) |
| AUTHORS | Mossessova, E., Gulbis, J. M. and Goldberg, J. |
| TITLE | Structure of the guanine nucleotide exchange factor Sec7 domain of |
| | human arno and analysis of the interaction with ARF GTPase |
| JOURNAL | Cell 92 (3), 415-423 (1998) |
| MEDLINE | 98135767 |
| PUBMED | 9476900 |
| REFERENCE | 7 (bases 1 to 1358) |
| AUTHORS | Frank, S., Upender, S., Hansen, S. H. and Casanova, J. E. |
| TITLE | ARNO is a guanine nucleotide exchange factor for ADP-ribosylation |
| | factor 6 |
| JOURNAL | J. Biol. Chem. 273 (1), 23-27 (1998) |
| MEDLINE | 98079021 |
| PUBMED | 9417041 |
| REFERENCE | 8 (bases 1 to 1358) |
| AUTHORS | Chardin, P., Paris, S., Antonny, B., Robineau, S., Beraud-Dufour, S., |
| | Jackson, C. L. and Chabre, M. |
| TITLE | A human exchange factor for ARF contains Sec7- and |
| | pleckstrin-homology domains |
| JOURNAL | Nature 384 (6608), 481-484 (1996) |
| MEDLINE | 97100951 |
| PUBMED | 8945478 |
| REFERENCE | 9 (bases 1 to 1358) |
| AUTHORS | Kolanus, W., Nagel, W., Schiller, B., Zeitlmann, L. , Godar, S., |
| | Stockinger, H. and Seed, B. |
| TITLE | Alpha L beta 2 integrin/LFA-1 binding to ICAM-1 induced by |
| | cytohesin-1, a cytoplasmic regulatory molecule |
| JOURNAL | Cell 86 (2), 233-242 (1996) |
| MEDLINE | 96319726 |
| PUBMED | 8706128 |
| COMMENT | REVIEWED REFSEQ: This record has been curated by NCBI staff. The |
| | reference sequence was derived from X99753.1 and U70728.1. |
| | On Oct. 18, 2000 this sequence version replaced gi: 8670547. |
| | Summary: Pleckstrin homology, Sec7 and coiled/coil domains 2 |
| | (PSCD2) is a member of the PSCD family. Members of this family have |
| | identical structural organization that consists of an N-terminal |
| | coiled-coil motif, a central Sec7 domain, and a C-terminal |
| | pleckstrin homology (PH) domain. The coiled-coil motif is involved |
| | in homodimerization, the Sec7 domain contains guanine-nucleotide |
| | exchange protein (GEP) activity, and the PH domain interacts with |
| | phospholipids and is responsible for association of PSCDs with |
| | membranes. Members of this family appear to mediate the regulation |
| | of protein sorting and membrane trafficking. PSCD2 exhibits GEP |
| | activity in vitro with ARF1, ARF3, and ARF6. PSCD2 protein is 83% |
| | homologous to PSCD1. |
| | Transcript Variant: This transcript (2) is missing 3 bp in the PH |
| | domain region, which results in a protein isoform missing a single |
| | glycine residue. |
| FEATURES | Location/Qualifiers |
| source | 1..1358 |
| | /organism=“Homo sapiens” |
| | /mol_type=“mRNA” |
| | /db_xref=“taxon:9606” |
| | /chromosome=“19” |
| | /map=“19q13.3” |
| gene | 1..1358 |
| | /gene=“PSCD2” |
| | /note=“synonyms: ARNO, CTS18.1, Sec7p-L” |
| | /db_xref=“GeneID:9266” |
| | /db_xref=“LocusID:9266” |
| | /db_xref=“MIM:602488” |
| CDS | 159..1358 |
| | /gene=“PSCD2” |
| | /note=“pleckstrin homology, Sec7 and coiled/coil domains |
| | 2; cytohesin 2; |
| | go_component: kinesin complex [goid 0005871] [evidence |
| | IEA]; |
| | go_component: membrane fraction [goid 0005624] [evidence |
| | TAS] [pmid 9417041]; |
| | go_component: plasma membrane [goid 0005886] [evidence |
| | TAS] [pmid 9417041]; |
| | go_function: ARF guanyl-nucleotide exchange factor |
| | activity [goid 0005086] [evidence TAS] [pmid 9417041]; |
| | go_function: guanyl-nucleotide release factor activity |
| | [goid 0019839] [evidence IEA]; |
| | go_process: actin cytoskeleton reorganization [goid |
| | 0007012] [evidence TAS] [pmid 9417041]; |
| | go_process: endocytosis [goid 0006897] [evidence TAS] |
| | [pmid 9417041]” |
| | /codon_start=1 |
| | /product=“pleckstrin homology, Sec7 and coiled/coil |
| | domains 2 isoform 2” |
| | /protein_id=“NP_004219.1” |
| | /db_xref=“GI:4758966” |
| | /db_xref=“GeneID:9266” |
| | /db_xref=“LocusID:9266” |
| | /db_xref=“MIM:602488” |
| | /translation=“MEDGVYEPPDLTPEERMELENIRRRKQELLVEIQRLREELSEAM |
| | SEVEGLEANEGSKTLQRNRKMAMGRKKFNMDPKKGIQFLVENELLQNTPEEIARFLYK |
| | GEGLNKTAIGDYLGEREELNLAVLHAFVDLHEFTDLNLVQALRQFLWSFRLPGEAQKI |
| | DRMMEAFAQRYCLCNPGVFQSTDTCYVLSFAVIMLNTSLHNPNVRDKPGLERFVAMNR |
| | GINEGGDLPEELLRNLYDSIRNEPFKIPEDDGNDLTHTFFNPDREGWLLKLGGRVKTW |
| | KRRWFILTDNCLYYFEYTTDKEPRGIIPLENLSIREVDDPRKPNCFELYIPNNKGQLI |
| | KACKTEADGRVVEGNHMVYRISAPTQEEKDEWIKSIQAAVSVDPFYEMLAARKKRISV |
| | KKKQEQP” |
| misc_feature | 195..320 |
| | /gene=“PSCD2” |
| | /note=“Region: Coiled-coil domain” |
| misc_feature | 336..887 |
| | /gene=“PSCD2” |
| | /note=“Sec7; Region: Sec7 domain. The Sec7 domain is a |
| | guanine-nucleotide-exchange-factor (GEF) for the pfam00025 |
| | family” |
| | /db_xref=“CDD:pfam01369” |
| misc_feature | 372..914 |
| | /gene=“PSCD2” |
| | /note=“Region: Sec7 domain” |
| misc_feature | 936..1283 |
| | /gene=“PSCD2” |
| | /note=“PH; Region: Pleckstrin homology domain. Domain |
| | commonly found in eukaryotic signalling proteins. The |
| | domain family possesses multiple functions including the |
| | abilities to bind inositol phosphates, and various |
| | proteins. PH domains have been found to possess inserted |
| | domains (such as in PLC gamma, syntrophins) and to be |
| | inserted within other domains. Mutations in Brutons |
| | tyrosine kinase (Btk) within its PH domain cause X-linked |
| | agammaglobulinaemia (XLA) in patients. Point mutations |
| | cluster into the positively charged end of the molecule |
| | around the predicted binding site for phosphatidylinositol |
| | lipids” |
| | /db_xref=“CDD:smart00233” |
| misc_feature | 942..1283 |
| | /gene=“PSCD2” |
| | /note=“Region: PH domain” |

| | | | | | | |
|---|---|---|---|---|---|---|
| 1 | ttccgaagga | agagtctttt | cagcgctgag | gactggcgct | gaggaggcgg | cggtggctcc |
| 61 | cggggcgttt | gagcgggctc | acccgagccg | cgggccaacg | cggatccagg | cccgactgcg |
| 121 | ggaccgcccc | ggattccccg | cgggccttcc | tagccgccat | ggaggacggc | gtttatgaac |
| 181 | ccccagacct | gactccggag | gagcggatgg | agctggagaa | catccggcgg | cggaagcagg |
| 241 | agctgctggt | ggagattcag | cgcctgcggg | aggagctcag | tgaagccatg | agcgaggtgg |
| 301 | aggggctgga | ggccaatgag | ggcagtaaga | ccttgcaacg | gaaccggaag | atggcaatgg |
| 361 | gcaggaagaa | gttcaacatg | gaccccaaga | aggggatcca | gttcttggtg | gagaatgaac |
| 421 | tgctgcagaa | cacacccgag | gagatcgccc | gcttcctgta | caagggcgag | gggctgaaca |
| 481 | agacagccat | cggggactac | ctgggggaga | gggaagaact | gaacctggca | gtgctccatg |
| 541 | cttttgtgga | tctgcatgag | ttcaccgacc | tcaatctggt | gcaggccctc | aggcagtttc |
| 601 | tatggagctt | tcgcctaccc | ggagaggccc | agaaaattga | ccggatgatg | gaggccttcg |
| 661 | cccagcgata | ctgcctgtgc | aaccctgggg | ttttccagtc | cacagacacg | tgctatgtgc |
| 721 | tgtccttcgc | cgtcatcatg | ctcaacacca | gtctccacaa | tcccaatgtc | cgggacaagc |
| 781 | cgggcctgga | gcgctttgtg | gccatgaacc | ggggcatcaa | cgagggcggg | gacctgcctg |
| 841 | aggagctgct | caggaacctg | tacgacagca | tccgaaatga | gcccttcaag | attcctgagg |
| 901 | atgacgggaa | tgacctgacc | cacaccttct | tcaacccgga | ccgggagggc | tggctcctga |
| 961 | agctgggggg | ccgggtgaaa | acgtggaagc | ggcgctggtt | tatcctcaca | gacaactgcc |
| 1021 | tctactactt | tgagtacacc | acggacaagg | agccccgagg | aatcatcccc | ctggagaatc |
| 1081 | tgagcatccg | agaggtggac | gacccccgga | aaccgaactg | ctttgaactt | tacatcccca |
| 1141 | acaacaaggg | gcagctcatc | aaagcctgca | aaactgaggc | ggacggccga | gtggtggagg |
| 1201 | gaaaccacat | ggtgtaccgg | atctcggccc | ccacgcagga | ggagaaggac | gagtggatca |
| 1261 | agtccatcca | ggcggctgtg | agtgtggacc | ccttctatga | gatgctggca | gcgagaaaga |
| 1321 | agcggatttc | agtcaagaag | aagcaggagc | agccctga | | |

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to PH domain variant nucleotide sequences can be prepared by standard synthetic techniques, e.g., using an automated DNA synthesizer.

In still another preferred embodiment, an isolated nucleic acid molecule of the present invention comprises a nucleotide sequence which is at least about 90%, 91%, 92%, 93%, 94%, 95%, 96%, 97%, 98%, 99%, 99.5% or more identical to the entire length of the nucleotide sequence shown in SEQ ID NO:2 or 4, or to the entire length of the PH domain of SEQ ID NO:2 or 4 (SEQ ID NO:5 and 6).

The invention further encompasses nucleic acid molecules that differ from the nucleotide sequence shown in SEQ ID NO:2 or 4 or the PH domain encoded by SEQ ID NO:2 or 4.

Variants of PH domains include both functional and non-functional proteins. Functional variants are amino acid sequence variants of proteins containing PH domains that maintain the ability to bind a ligand or substrate (e.g., phosphoinostide). Functional variants will typically contain only conservative substitution of one or more amino acids of SEQ ID NO:1 or 3, or substitution, deletion or insertion of non-critical residues in non-critical regions of the protein. In specific embodiments, the PH variants will have insertions, deletions, or substitutions in the loop regions that connect the β strands.

Non-functional variants are amino acid sequence variants of PH domain containing proteins that do not have the ability to either bind an PH domain ligand or substrate (e.g., phosphoinostide). Non-functional variants will typically contain a non-conservative substitution, a deletion, or insertion or premature truncation of the amino acid sequence of SEQ ID NO:1 or 3, or a substitution, insertion or deletion in critical residues or critical regions.

Accordingly, another aspect of the invention pertains to nucleic acid molecules encoding PH domain containing proteins that contain changes in amino acid residues that are not essential for activity. Such PH domain containing proteins differ in amino acid sequence from SEQ ID NO:1 or 3, yet retain biological activity. In one embodiment, the isolated nucleic acid molecule comprises a nucleotide sequence encoding a protein, wherein the protein comprises an amino acid sequence at least 90%, 91%, 92%, 93%, 94%, 95%, 96%, 97%, 98%, 99%, 99.5% or more identical to SEQ ID NO:1 or3.

A nucleic acid molecule encoding an variant PH domain containing protein, can be created by introducing one or more nucleotide substitutions, additions or deletions into the nucleotide sequence of SEQ ID NO:2 or 4, such that one or more amino acid substitutions, additions or deletions are introduced into the encoded protein. Mutations can be introduced into SEQ ID NO:2 or 4, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a PH domain containing protein is preferably replaced with another amino acid residue from the same side chain family. Alternatively, in another embodiment, mutations can be introduced randomly along all or part of a PH domain containing coding sequence, such as by saturation mutagenesis, and the resultant mutants can be screened for PH domain biological activity to identify mutants that retain activity. Following mutagenesis of SEQ ID NO:2 or 4, the encoded protein can be expressed recombinantly and the activity of the protein can be determined.

Variants

The instant invention provides variants of PH domain containing proteins. In one embodiment the variants are variants of SEQ ID NO:1 or 3. In another embodiment the variants are variants of the PH domain of SEQ ID NO: 1 or 3 (SEQ ID NO:5 or 6). The variants are further described in the Proteins section above.

Variants polypeptides of the invention are variants of GAC family polypeptides that have altered binding properties compared to polypeptides with the wild-type sequence due to one or more insertion, deletion or substitution in the PH domain. In one embodiment, the variant polypeptide binds to one or more natural ligand with increased affinity. Alternatively, the variant polypeptide of the invention binds to one or more natural ligand with decreased affinity. In a third embodiment, the variant polypeptide of the invention binds to one ligand with increased or decreased affinity while binding to another ligand with decreased affinity. In a fourth embodiment, the variant polypeptide of the invention binds to one ligand with increased or decreased affinity while binding to another ligand with increased affinity. In a fifth embodiment, the variant polypeptide of the invention binds to one ligand with increased or decreased affinity while not changing the affinity for another ligand. The affinity of the variant can be measured as described herein. In certain embodiments the affinity can be changed by 10, 50, 100, 500, 1000, or 10000 times.

In specific embodiments the variants of the invention have inserted, deleted or substituted residues in the loops that connect the β strands. In certain embodiments, the insertions are one or more glycine residues.

In another embodiment the variant polypeptide has altered specificity for one or more ligands. In one embodiment, the variant polypeptide may be able to selectively bind to one phosphoinositide while not binding to other, whereas the wild-type sequence bind promiscuously to multiple phosphoinositides.

Assays

The invention provides variants of GAC family proteins. Once GAC family protein variants are made and expressed, the following assays can be used to test their ability to interact with ligand.

In one embodiment, the assay of the present invention is a cell-free assay in which a GAC family polypeptide (e.g., protein or variant) or biologically active portion thereof is contacted with a compound, e.g., a phosphoinositide, and the ability of the compound to bind to the GAC polypeptide is determined. Preferred biologically active portions of the GAC family polypeptides to be used in assays of the present invention include fragments which contain a PH domain. Binding of the test compound to the GAC family polypeptide can be determined either directly or indirectly as described herein. In a preferred embodiment, the assay includes contacting the GAC family polypeptide or biologically active portion thereof with a first compound which binds to a GAC family polypeptide to form an assay mixture, contacting the assay mixture with a second compound, and determining the ability of the second compound to interact with a GAC family polypeptide, wherein determining the ability of the second compound to interact with a GAC family polypeptide comprises determining the ability of the second compound to preferentially bind to a GAC family polypeptide or biologically active portion thereof as compared to the known compound. First and second compounds are, for example, different phosphoinositides.

In another embodiment, the assay is a cell-free assay in which a GAC family polypeptide, variant thereof or biologically active portion thereof is contacted with a compound and the ability of the compound bind to the GAC family polypeptide, variant thereof or biologically active portion thereof is determined. Determining the ability of the test compound to modulate an activity of a GAC family polypeptide or a variant thereof, e.g., the ability to participate in cell survival, membrane trafficking, insulin regulation, cell adhesion, cell migration and cytoskeletal dynamics can be accomplished, for example, by determining the ability of the GAC family polypeptide, or variant thereof to bind to a GAC family polypeptide target molecule, e.g., a phosphoinositide, by one of the methods described above for determining direct binding. Determining the ability of a GAC family polypeptide, or variant thereof, to bind to a GAC family protein target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a GAC family polypeptide can be accomplished by determining the ability of the GAC family polypeptide or variant thereof to further modulate the activity of a downstream effector of a GAC family protein target molecule.

In yet another embodiment, the cell-free assay involves contacting a GAC family polypeptide , variant thereof or biologically active portion thereof with a known compound which binds the GAC family polypeptide to form an assay mixture, contacting the assay mixture with a compound, and determining the ability of the test compound to interact with the GAC family polypeptide, wherein determining the ability of the test compound to interact with the GAC family polypeptide comprises determining the ability of the GAC family polypeptide to preferentially bind to or modulate the activity of a GAC family target molecule.

The above cell-free and cell based assays exemplify the utility of the variant polypeptides of the invention. In particular, variant polypeptides of the invention can be used in any assay suitable for detecting interaction between ligands (e.g., phophotidylinositides) and PH domains, for example, those described above, as well as other suitable art-recognized assays. Direct assays (e.g., direct binding and/or activity assays) as well as indirect assays (e.g., assays for downstream effects, for example, signaling effects or resulting cellular effects) are within the scope of the invention. An exemplary use for the variant polypeptides of the invention is in the detection of specific phosphotidylinositides. For example, a variant polypeptide having an altered specificity for a given phosphotidylinositide can be used as a detection reagent for said phosphotidylinositide, preferably in a manner that excludes detection of other phosphotidylinositides. Alternatively, variant polypeptides of the invention can be used as control reagents, for example, as positive, negative or specificity control reagents. Other uses for the variant polypeptides of the invention will be apparent from the following Examples and claims.

Exemplification

The invention is further illustrated by the following examples which should not be construed as limiting.

### EXAMPLE

### Example 1

### Ability of Variant Grp1 and ARNO to Bind IP

Variant Grp1 and ARNO polypeptides were constructed. Each variant was tested for the ability to bind to the natural IP ligand.

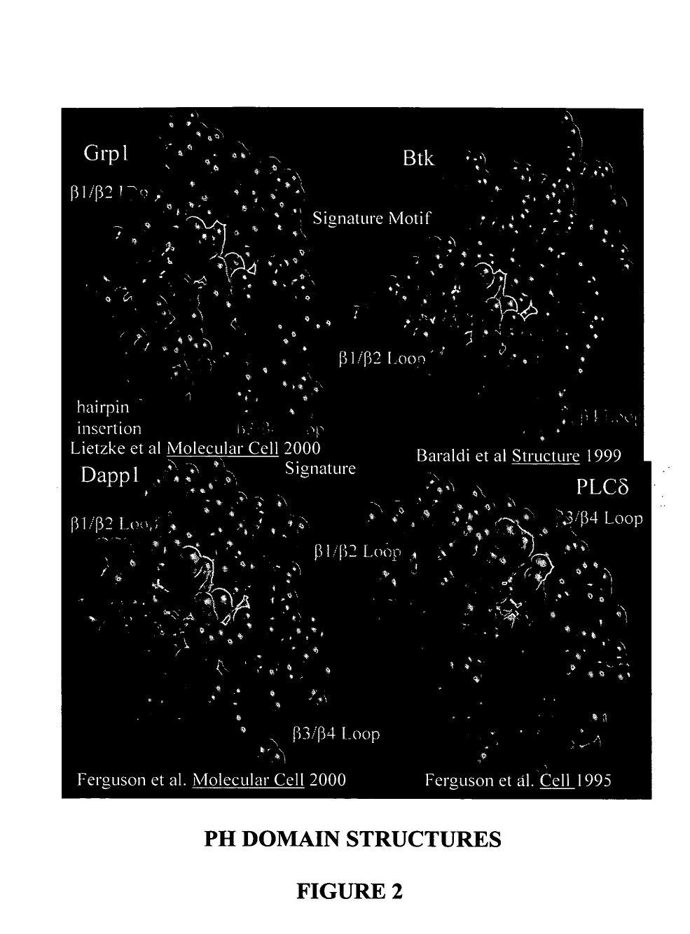

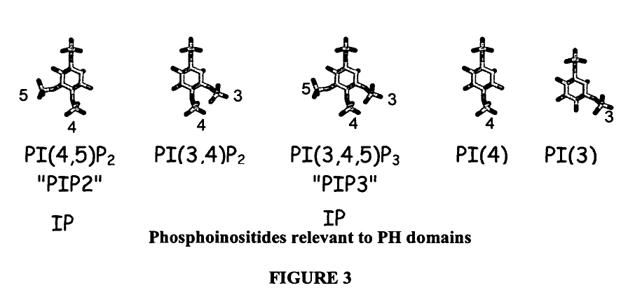

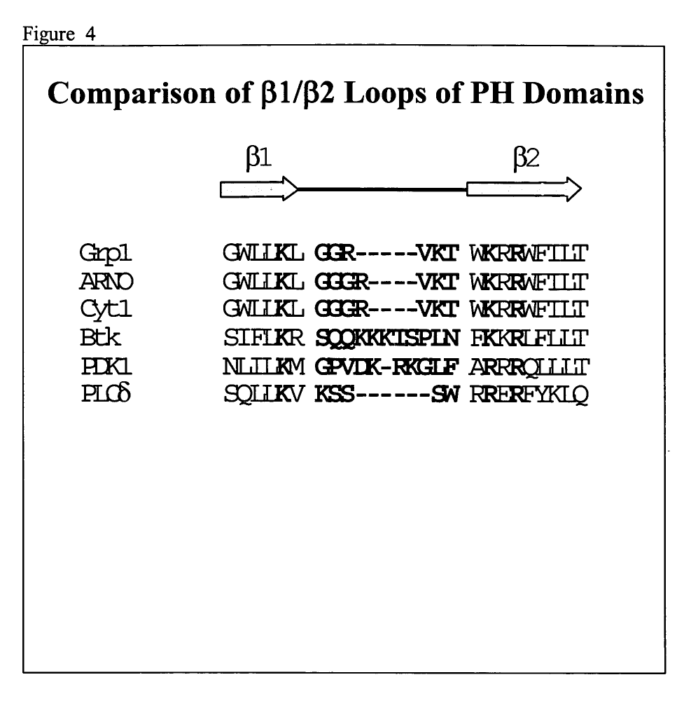

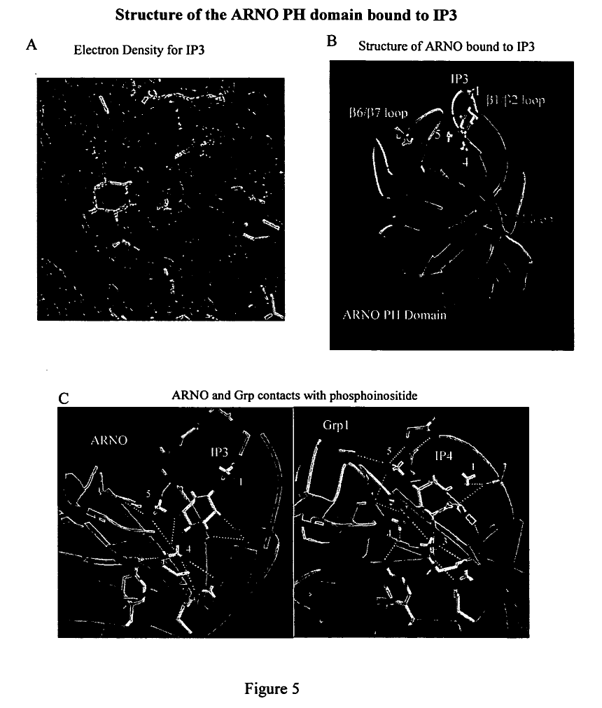

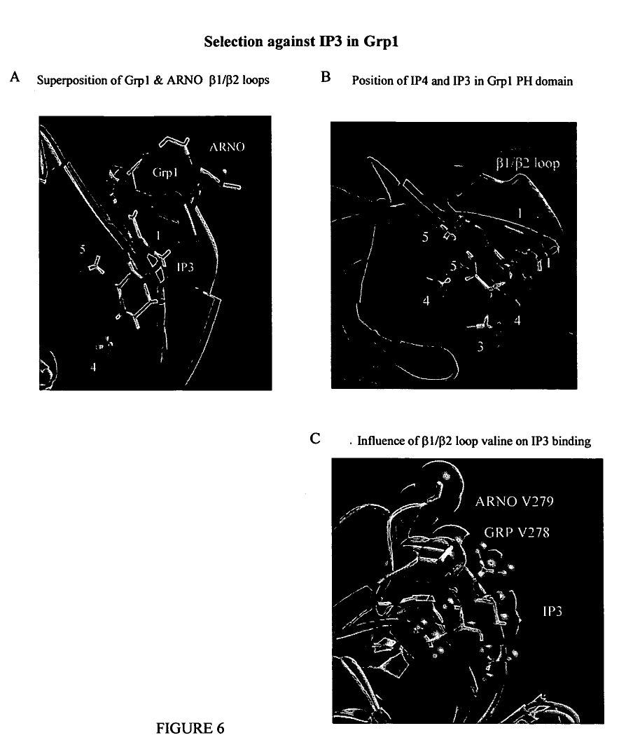

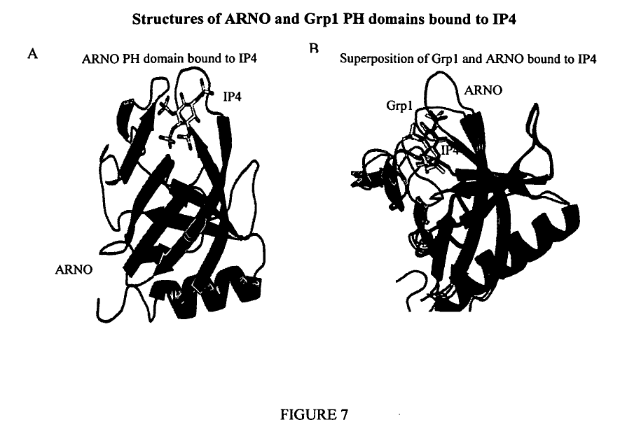

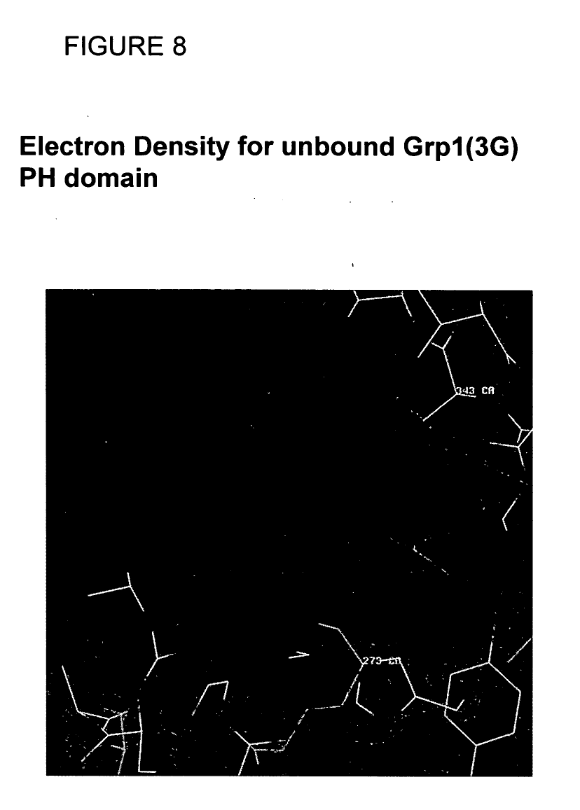

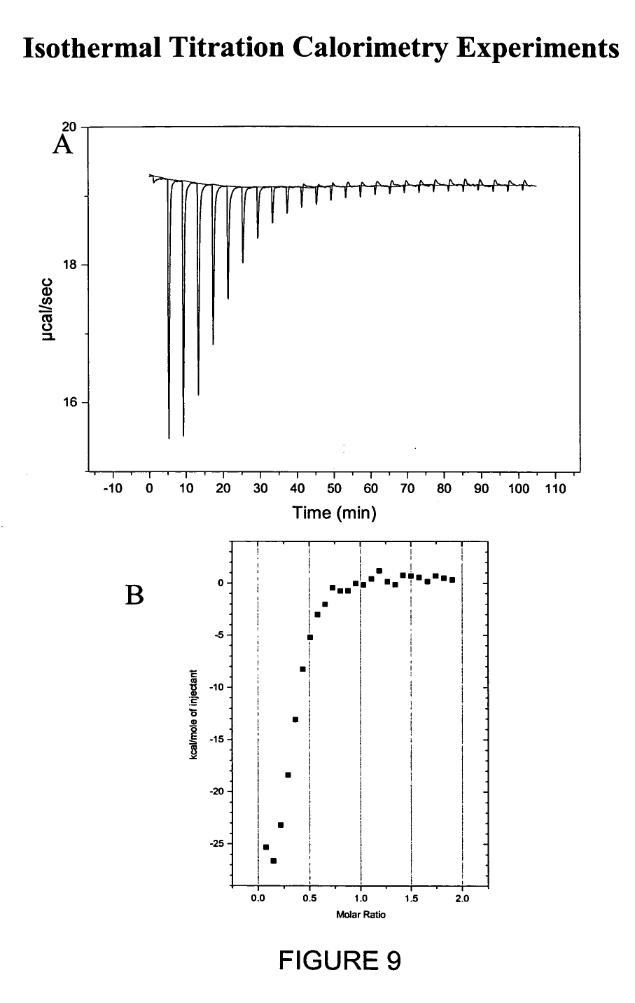

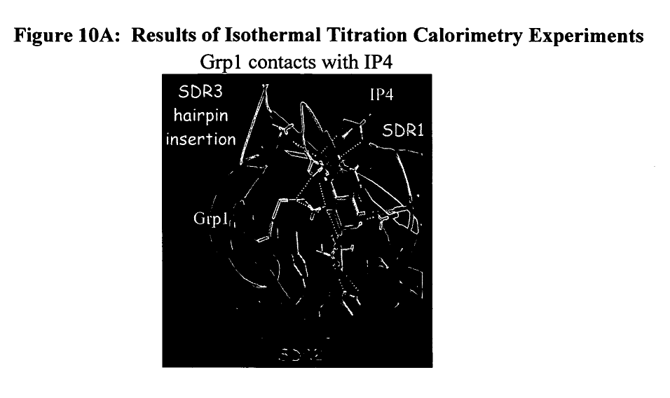

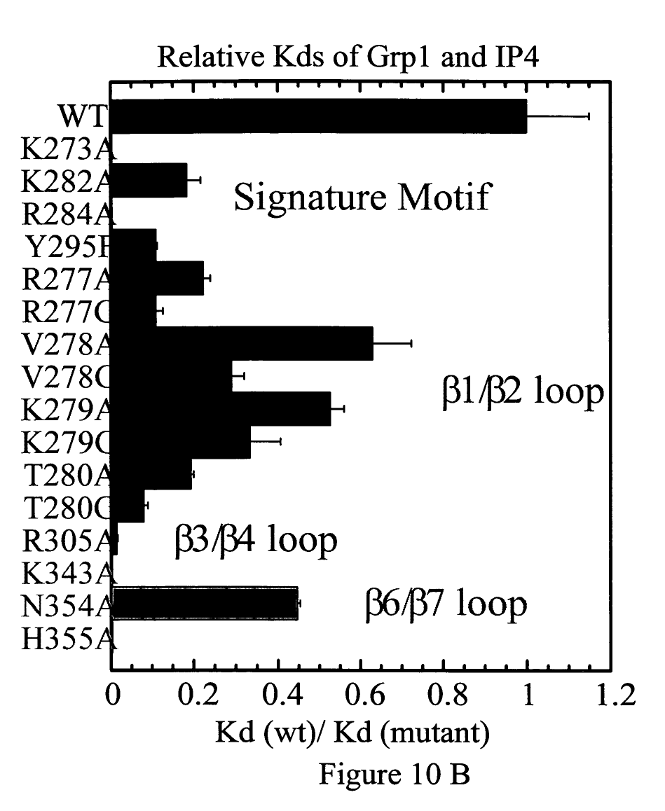

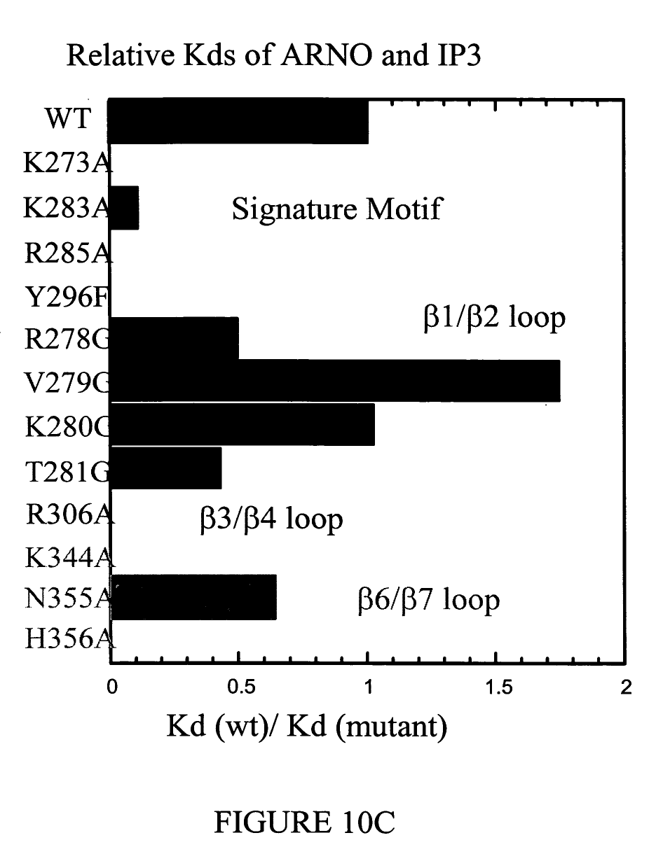

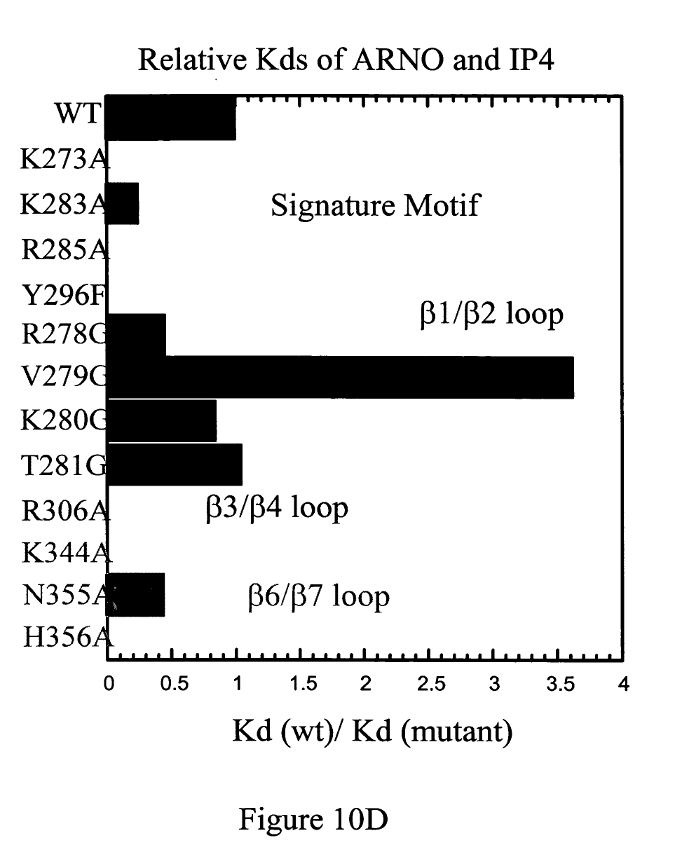

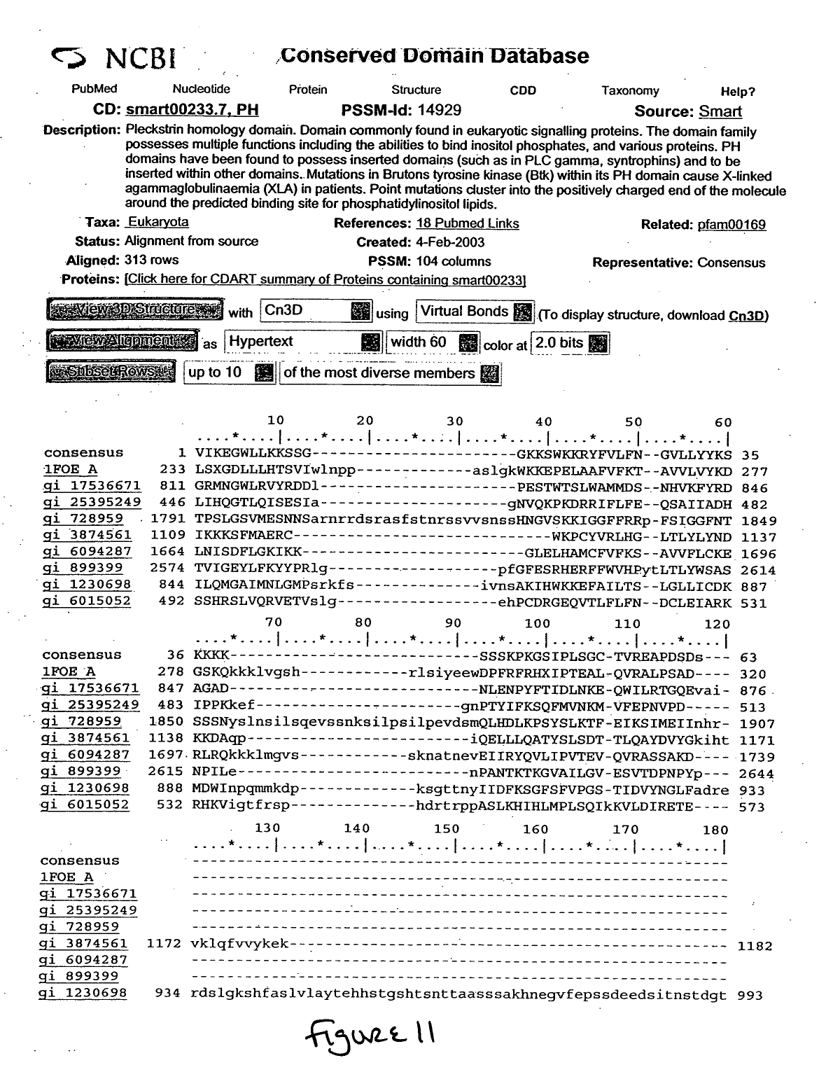

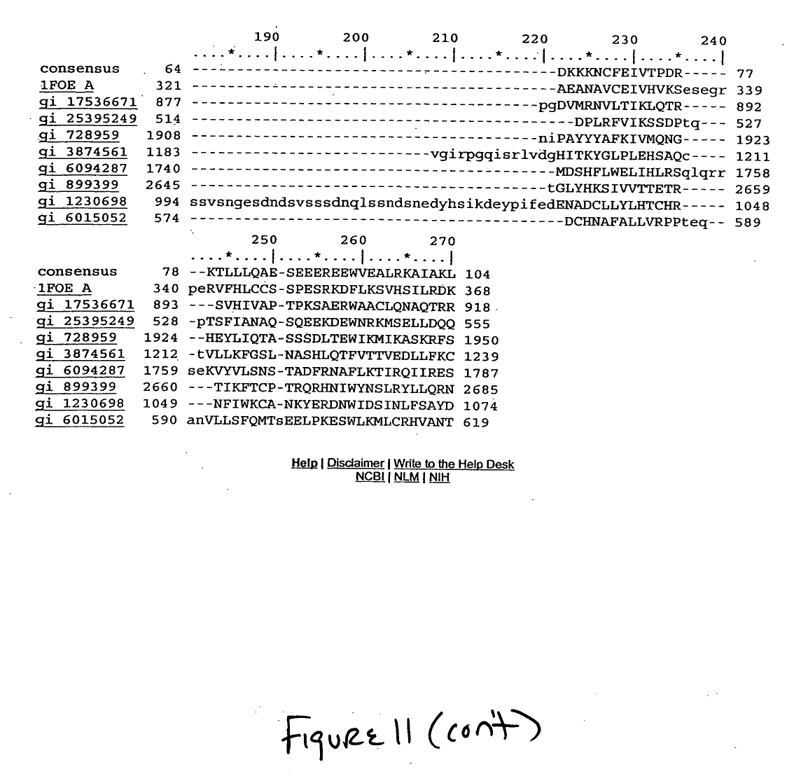

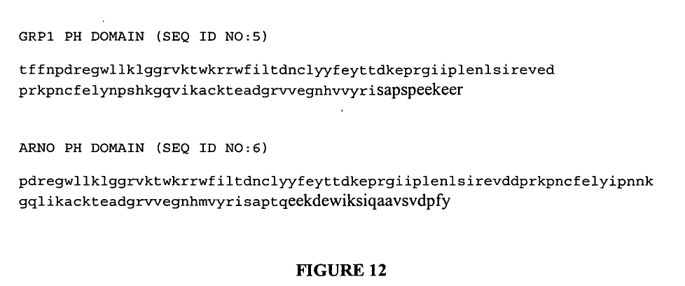

Constructs of GST tagged Grp1(2G) were titrated with IP4. The dissociation constant (Kd) for each interaction was determined and compared to the wild type. The relative Kds are plotted for each Grp1 mutant in FIG. 2A.

Constructs of GST tagged ARNO (3G) were titrated with IP3 or IP4. The dissociation constant (Kd) for each interaction was determined and compared to the wild type. The relative Kds are plotted for each ARNO mutant in FIGS. 2B and C, respectively. The V279G mutant bound IP3 and IP4 with higher affinity than wild type.

### Example 2

### Determination of Alternate Binding Modes of PtdIns(4,5)P2 and PtdIns(3,4,5)P3

The crystal structures of the 3G variant of the ARNO PH domain bound to the head groups of PtdIns(4,5)P2 and PtdIns(3,4,5)P3 has been determined. The structures reveal two distinct modes of binding, with different head group orientations and different networks of interactions with amino acid residues in the binding site. Although the orientation of the PtdIns(3,4,5)P3 head group is nearly identical to that observed previously for the 2G Grp1 PH domain, the orientation and mode of PtdIns(4,5)P2 binding to the 3G ARNO PH domain is entirely novel. On the basis of the structural data, a systematic, quantitative, comprehensive mutational analysis of all residues, both conserved and non-conserved, that mediate interactions with the phosphoinositide head groups as well as other residues in the adjacent β1/β2 loop that do not contact the head group directly has been preformed. These studies reveal that some of the variant Grp1 and ARNO PH domains, differing by single amino acid substitutions, have altered specificities as well as affinities for phosphoinositide head groups. The extensive structural, mutational, and binding data provide the information necessary to design and optimize variant Grp1/ARNO/Cytohesin family PH domains with altered binding affinity and/or specificity. Such engineered PH domains can be used as biochemical reagents for detection of specific phosphoinositides in in vitro and/or cell based assays. In addition, a number of stable mutants of the Grp1 and ARNO PH domains have been identified, which are unable to bind phosphoinositides. These mutant proteins can be used as key controls to determine the extent of and correct for non-specific background binding. It is possible to introduce mutations that reduce non-specific background binding without affecting the affinity and specificity for phosphoinositides. With respect to practicality and feasibility, it has been: i) established that the Grp1 and ARNO PH domains can be efficiently prepared as GST-fusion proteins in high yield (20 mg/L of E. coll culture) and purity (>95%) in a single purification step; ii) determined that these GST fusion proteins behave homogenously and bind phosphoinositide head groups with 1:1 stoichiometry; and iii) determined that the GST fusions of Grp1 are highly tolerant of non-conservative amino acid substitutions within and adjacent to the head group binding site such that a variety of engineered Grp1 PH domains can be reliably produced in high yield and purity. Finally, given the efficiency/reliability of expression and purification, the production of GST fusions of wild type as well as variant Grp1 and ARNO PH domains is readily scaleable.

This is the first and currently the only observation of different binding modes for different head groups in the context of the same PH domain. Furthermore, it has been shown, at least in the case of the head group of PtdIns(3,4,5)P3, that the mode of binding is not altered by structural changes in the P1/P2 loop that dramatically alter specificity. It has further been shown that non-naturally occurring single amino acid substitutions, as well as naturally occurring single amino acid substitutions, alter both the affinity and specificity for phosphoinoisitides. Moreover, it has been demonstrated that the Grp1 and ARNO PH domains are amenable to crystallization in multiple forms including the unliganded form (2G and 3G Grp1), the complex with the head group of PtdIns(4,5)P′2 (3G ARNO), and the complex with the head group of PtdIns(3,4,5)P3 (2G Grp1 and 3G ARNO). In addition, it has been established that the phosphoinositide binding properties of Grp1 family PH domains are independent of the nature of the N-terminal fusion tag (GST or hexahistidine). Thus, the Grp1 family PH domains are uniquely suitable for structure based engineering of PH domains with novel affinities and specificities for phosphoinositides.

The structure of the ARNO PH domain reveals a novel mode of phosphoinositide binding. ARNO binds PtdIns(4,5)P2 in a rotated orientation and position when compared to previously characterized PH domains. The inositol ring of PIP2 is bound in a similar orientation to PIP3 in the Grp1 and Btk PH domains. However, the phosphates make different contacts than the highly homologous Grp1 . The 4-phosphate is contacted by the pocket that would recognize the 3-phosphate in Grp1. The 5-phosphate is contacted by the pocket that would recognize the 4-phosphate. Meanwhile, the residues that would make contact with the 5-phosphate in Grp1 are not contacting the ligand. The extra glycine in the β1/β2 loop of ARNO creates a longer loop that can accommodate PIP3 or PIP2 with little variation in specificity. However, the shorter loop in Grp1 brings a valine group too close to the inositol ring of PIP2 for a favorable interaction. This may explain the strong specificity Grp1 exhibits for PIP3 over PIP2. The presence of sulfate ions in the unliganded Grp1 (3G) structure in place of the phosphates suggest a preset placement for recognizing phosphoinositides in a limited binding orientation. Testing phosphoinositide binding in mutants of the Grp 1 and ARNO PH domains using isothermal calorimetry validate the necessity of certain residues for phosphoinositide binding. The presence of valine in the C terminus of the β1/β2 loop and a third glycine in the N terminus of the β1/β2 loop strongly affect phosphoinositide recognition.

References

1. T. F. Franke, D. R. Kaplan, L. C. Cantley, A. Toker, Science 275, 665-8 (Jan. 31, 1997).
2. H. W. Chang et al., Science 276, 1848-50 (Jun. 20, 1997).
3. T. Maehama, J. E. Dixon, J: S/o7: Cfew. 273, 13375-8 (May 29, 1998).
4. V. Stambolic et al, Cell 95, 29-39 (Oct. 2, 1998).
5. B. Vanhaesebroeck et al, Annu. Rev. Biochem. 70, 535-602 (2001).
6. T. Maehama, G. S. Taylor, J. E. Dixon, Annu. Rev. Biochem. 70, 247-79 (2001).
7. M. A. Lemmon, K. M. Ferguson, Biochem. J. 350 Pt 1, 1-18 (Aug. 15, 2000).
8. M. A. Lemmon, K. M. Ferguson, Biochem. Soc. Trans. 29,377-84 (August 2001).
9. M. A. Lemmon, K. M. Ferguson, C. S. Abrams, FEES Lett. 513, 71-6 (Feb. 20, 2002).
10. M. Tyers et al, Nature 333, 470-3 (Jun. 2, 1988).
11. A. Musacchio, T. Gibson, P. Rice, J. Thompson, M. Saraste, Trends Biochem. Sci. 18, 343-8 (September 1993).
12. R. J. Haslam, H. B. Koide, B. A. Hemmings, Nature 363, 309-10 (May 27, 1993).
13. B. J. Mayer, R. Ren, K. L. Clark, D. Baltimore, Cell 73, 629-30 (May 21, 1993).
14. J. C. Venter etal, Science 291, 1304-51 (Feb. 16, 2001).
15. E. S. Lander etal, Nature 409, 860-921 (Feb. 15, 2001).
16. M. A. Lemmon, K. M. Ferguson, R. O'Brien, P. B. Sigler, J. Schlessinger, Proc. Nail. Acad. Sci. U.S.A. 92, 10472-6 (Nov. 7, 1995).
17. L. E. Rameh et al., J. Biol. Chem. 272, 22059-66 (Aug. 29, 1997).
18. J. M. Kavran et al., J. Biol. Chem. 273, 30497-508 (Nov. 13, 1998).
19. S. J. Isakoff et al., Embo J. 17, 5374-87 (Sep. 15, 1998).
20. M. J. Rebecchi, S. Scarlata, Amu. Rev. Biophys. Biomol. Struct. 27, 503-28 (1998).
21. N. Blomberg, E. Baraldi, M. Sattler, M. Saraste, M. Nilges, Structure Fold. Des. 8, 1079-87 (Oct. 15, 2000).
22. K. M. Ferguson et al., Mol. Cell 6, 373-84 (August 2000).
23. S. E. Lietzke et al, Mol. Cell 6, 385-94 (August 2000).
24. D. K. Worthylake, K. L. Rossman, J. Sondek, Nature 408, 682-8 (Dec. 7, 2000).
25. C. C. Thomas, S. Dowler, M. Deak, D. R. Alessi, D. M. van Aalten, Biochem. J. 358, 287-94 (Sep. 1, 2001).
26. J. T. Snyder et al., Nat. Struct. Biol. 9, 468-75 (June 2002).
27. K. L. Rossman et al., Embo J. 21, 1315-26 (Mar. 15, 2002).
28. C. C. Thomas, M. Deak, D. R. Alessi, D. M. van Aalten, Curr. Biol. 12, 1256-62 (M 23, 2002).
29. M. J. Macias et al, Nature 369, 675-7 (Jun. 23, 1994).
30. N. Blomberg, E. Baraldi, M. Nilges, M. Saraste, Trends Biochem. Sci. 24, 441-5 (November 1999).
31. K. Salimetal., Embo J. 15, 6241-50 (Nov. 15, 1996).
32. M. Fukuda, T. Kojima, H. Kabayama, K. Mikoshiba, J. Biol. Chem. 271, 30303-6 (Nov. 29, 1996).
33. J. K. Klarlund et al, Science 275, 1927-30 (Mar. 28, 1997).
34. M. Freeh et al, J. Biol Chem. 272, 8474-81 (Mar. 28, 1997).
35. V. R. Rao et al, J. Biol Chem. 274, 37893-37900 (1999).
36. S. Dowler, R. A. Currie, C. P. Downes, D. R. Alessi, Biochem. J. 342 (Pt 1), 7-12 (Aug. 15, 1999).
37. M. Ogasawara et al., J. Biol. Chem. 275, 3221-30 (Feb. 4, 2000).
38. J. K. Klarlund, W. Tsiaras, J. J. Holik, A. Chawla, M. P. Czech, J. Biol. Chem. 275, 32816-21 (Oct. 20, 2000).
39. K. M. Ferguson, M. A. Lemmon, J. Schlessinger, P. B. Sigler, Cell 83, 1037-46 (Dec. 15, 1995).
40. E. Baraldi et al, Structure Fold. Des. 1, 449-60 (Apr. 15, 1999).
41. H. Yagisawa et al., J. Biol. Chem. 273, 417-24 (Jan. 2, 1998).
   Equivalents

Those skilled in the art will recognize, or be able to ascertain using no more than routine experimentation, many equivalents to the specific embodiments and methods described herein. Such equivalents are intended to be encompassed by the scope of the following claims.

Incorporation by Reference

All publications, patent applications, patents, and other references mentioned herein are incorporated by reference in their entirety.

1. A polypeptide comprising a variant pleckstrin homology (PH) domain wherein said variant domain has an altered specificity for binding to a phosphatidylinositide molecule.

2. The polypeptide of claim 1, wherein said polypeptide has increased binding specificity for a phosphatidylinositide molecule to which the PH domain naturally binds.

3. The polypeptide of claim 1, wherein said polypeptide has decreased binding specificity for a phosphatidylinositide molecule to which the PH domain naturally binds.

4. The polypeptide of claim 1, wherein said polypeptide has increased binding specificity for a phosphatidylinositide molecule to which the PH domain naturally does not bind.

5. The polypeptide of claim 1, wherein said polypeptide has decreased binding specificity for a phosphatidylinositide molecule to which the PH domain naturally does not bind.

6. The polypeptide of claim 1, wherein said phosphatidylinositol molecule is phosphatidylinositol-3,4,5 (PI-3,4,5)P3.

7. The polypeptide of claim 1, wherein said phosphatidylinositol molecule is phosphatidylinositol-4,5 (PI-4,5)P2.

8. The polypeptide of claim 2, wherein said variant PH domain has an increased specificity for binding to PI-3,4,5P3.

9. The polypeptide of claim 2, wherein said variant PH domain has a decreased specificity for binding to PI-3,4,5P2.

10. The polypeptide of claim 3, wherein said variant PH domain has an increased specificity for binding to PI-4,5P2.

11. The polypeptide of claim 3, wherein said variant PH domain has a decreased specificity for binding to PI-4,5P2.

12. The polypeptide of claim 1, wherein said polypeptide has an amino acid insertion in a loop that connects two beta strands within the PH domain.

13. The polypeptide of claim 1, wherein said polypeptide has an amino acid deletion in a loop that connects two beta strands within the PH domain.

14. The polypeptide of claim 1, wherein said polypeptide has an amino acid substitution in a loop that connects two beta strands within the PH domain.

15. The polypeptide of claim 1, wherein said polypeptide has at least one glycine residue inserted in the β1/β2 loop as compared to the wild-type sequence.

16. The polypeptide of claim 15, wherein said polypeptide has two glycine residues inserted.

17. The polypeptide of claim 15, wherein said polypeptide has three glycines residues inserted.

18. The polypeptide of claim 1, wherein said variant comprises an amino acid substitution in a residue within the PH domain that does not contact the head group of said phosphatidylinositol.

19. The polypeptide of claim 1, wherein said PH domain is from a Grp1/ARNO/Cytohesin family peptide.

20. The polypeptide of claim 1, wherein said polypeptide has a 10 fold higher specificity for a given phosphatidylinositide molecule than the wild-type polypeptide.

21. The polypeptide of claim 1, wherein said polypeptide has a 100 fold higher specificity for a given phosphatidylinositide molecule than the wild-type polypeptide.

22. The polypeptide of claim 1, wherein said polypeptide has a 1000 fold higher specificity for a given phosphatidylinositide molecule than the wild-type polypeptide.

23. A polypeptide comprising a variant PH domain wherein said variant increases the affinity of the PH domain for one ligand while not changing the affinity for a second ligand.

24. A polypeptide comprising a variant PH domain wherein said variant increases the affinity of the PH domain for one ligand while decreasing the affinity for a second ligand.

25. A polypeptide comprising a variant PH domain wherein said variant increases the affinity of the PH domain for one ligand while increasing the affinity for a second ligand.

26. The polyeptide of anyone of claims 23-25, wherein said second ligand is a natural ligand of the PH domain.

27. The polyeptide of anyone of claims 23-25, wherein said second ligand is not a natural ligand of the PH domain.

28. A polypeptide comprising a variant PH domain wherein said variant decreases the affinity of the PH domain for one ligand while not changing the affinity for a second ligand.

29. A polypeptide comprising a variant PH domain wherein said variant decreases the affinity of the PH domain for one ligand while decreasing the affinity for a second ligand.

30. A polypeptide comprising a variant PH domain wherein said variant decreases the affinity of the PH domain for one ligand while increasing the affinity for a second ligand.

31. The polypeptide of anyone of claims 28-30 wherein said second ligand is a natural ligand of the PH domain.

32. The polyeptide of anyone of claims 28-30, wherein said second ligand is not a natural ligand of the PH domain.

33. The polypeptide of any one of claims 23-25 and 28-30, wherein said polypeptide has more than one amino acid substitutions, insertions or deletions.

34. The polypeptide of claim 33, wherein said polypeptide is used as a negative control for binding a specific ligand.

35. The polypeptide of claim 33, wherein said polypeptide has increased affinity for a substrate.

36. The polypeptide of claim 33, wherein said polypeptide has modified specificity for ligands.

37. A variant GRP1 polyeptide selected from the group consisting of K273A, K282A, R284A, Y295F, R277A, R277C, V278A, V278C, K279A, K279C, T280A, T280C, R305A, K343A, N354A, and H355A of SEQ ID NO:1.

38. A variant GRP1 polyeptide having one or more of the following substitutions: of K273A, K282A, R284A, Y295F, R277A, R277G, V278A, V278C, K279A, K279G, T280A, T280G, R305A, K343A, N354A, and/or H355A of SEQ ID NO:1.

39. A variant ARNO polyeptide selected from the group consisting of K273A, K283A, R285A, Y296F, R278G, V279G, K280G, T281G, R306A, K344A, N355A, and H356A of SEQ ID NO:3.

40. A variant ARNO polyeptide having one or more of the following substitutions: K273A, K283A, R285A, Y296F, R278G, V279G, K280G, T281G, R306A, K344A, N355A, and/or H356A of SEQ ID NO:3.

41. A nucleic acid molecule that encodes the polypeptide of any one of claims 1, 23-25 or 28-30.

42. The nucleic acid molecule of claim 41, wherein said nucleic acid molecule is in a vector.

43. The nucleic acid molecule of claim 42, wherein said vector is an expression vector.

44. Use of the variant of any one of claims 1, 23-25, or 28-30 to selectively detect the presence of a specific phosphatidylinositide.

45. Use of the variant of any one of claims 1, 23-25, or 28-30 as a control in an assay to detect the presence of a specific phosphatidylinositide.

46. The use of claim 44, wherein said phosphatidylinositol molecule is phosphatidylinositol-3,4,5 (PI-3,4,5)P3.

47. The use of claim 44, wherein said phosphatidylinositol molecule is phosphatidylinositol-4,5 (PI-4,5)P2.


SEQUENCE LISTING (5 sequences)

SEQ ID NO: 1 - protein, 399 aa, Homo sapiens
MDEDGGGEGGGVPEDLSLEEREELLDIRRRKKELIDDIERLKYEIAEVMTEIDNLTSVEESKTTQRNKQIAMGRKKFNMDPKKGIQFLIENDLLQSSPEDVAQFLYKGEGLNKTVIGDYLGERDEFNIKVLQAFVELHEFADLNLVQALRQFLWSFRLPGEAQKIDRMMEAFASRYCLCNPGVFQSTDTCYVLSFAIIMLNTSLHNHNVRDKPTAERFIAMNRGINEGGDLPEELLRNLYESIKNEPFKIPEDDGNDLTHTFFNPDREGWLLKLGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASISRDPFYDMLATRKRRIANKK

SEQ ID NO: 2 - DNA, 4482 bp, Homo sapiens
tgaggagccgcccggtcgcctgcgcgctccctccggcggcgtccccagcccgcggcccctctgctgccggcccccggctcgccggctgcgggagtggcctcaagatggatgaagacggcggcggcgagggtggtggcgtgcctgaagacctctcattagaagagagagaagaacttctagacattcgtcgaagaaaaaaggaacttattgatgacattgagaggctgaaatatgaaattgcagaggtgatgacagagatcgacaatctaacttccgtagaggagagcaaaacgactcagaggaacaaacagatagccatgggaagaaagaaattcaacatggatcccaaaaagggaattcagtttctaatagaaaatgacctgctacagagttccccagaagacgtcgcccagttcctttataaaggagaaggcctaaataagaccgtcattggggactacctgggtgaaagggatgaatttaatattaaagttcttcaagcctttgttgaactccatgagtttgctgatctcaaccttgtacaagccttaaggcagttcttatggagcttcaggctgcccggggaggcgcagaagattgatcgcatgatggaggctttcgcttctcgctactgcctgtgcaaccccggggtcttccagtccacagacacgtgctacgtgctgtcattcgccatcatcatgctcaacaccagcctccacaaccacaacgtgcgtgacaagcccacggcagaacggttcatcgccatgaaccgcggcatcaacgagggcggggacctccctgaggagctgctgaggaatttgtatgagagcattaagaacgagccatttaagatcccggaggacgacgggaacgacctgacccacaccttcttcaaccccgaccgcgagggctggctcctgaagctgggagggcgtgtgaagacctggaagcgccggtggttcatcctgaccgataactgcctctattactttgaatacacaacagataaggagcccaggggaatcatcccgttggaaaacctcagcatcagggaggtggaggacccccggaaacccaactgttttgagctctacaatcccagccacaaagggcaggtcatcaaggcctgtaagactgaggccgacggccgcgtggtagaggggaaccatgtggtgtaccggatctcagccccgagcccggaggagaaggaggagtggatgaaatccatcaaagccagtatcagcagagatcccttctatgacatgttggcaacgaggaaacgaaggattgccaataaaaaatagctttcctggctaaaagacccaggtaaaagacccaaccccagcagaaagacaccgcgggcggcccctccgcggaaggcgtggcagggaggcagtcgccctgcggtgcaagctgctgctccagagcataccgtggcccaggtggtatccccaaggcctcgtgccgtggctggggtcctgggaggtggtcgccctgcagtgcaagctgctgctccagagcgtaccgtggcccagactgatcctcgaggcctcctgccgtggctggggtcatggtcggctgcgcatgtccagaagcatttccttcctgcgaccatcccggcgcccctagggggagaagccaggacagcagcttccgctgtctccacagcagacacgggacggattccacagacgggagcctcattcgtaccatgccaaacgcattcactcggggcagtattaaccgttctagaaagccactgttttatagcaaaacaggaaaggaaaagctaccagttttttattcagaatttttctcagatatataggattatagcttttatatgcctttttatattctgaaattataacaaaagatactttctaacagtagtatttttagaatggcagctataaagttaactcctggacacaagtatatactgtgcactgaaaaaatatccatctacacagcacccaaggggagggctgggggcaccggcacgggggcagcgtgcagccctgccctgtcaggctgtcagacaagccccggggggcagcaggtgggctcgggacgggctgggggagggacggccatggcacttgggggctccagggtgactcccatgaggcctcccttcaaccaggctttttggccccacaaatactttaagcaaatcattaaaattataacagttaatggtttgggggtgtttaggctgtaactgctaactcctaggaaacagccttttccctggacacagatggtccatacgctgagccacgtgaaactgctgatgttttgtttagatgcacacacatggcagcgtttcatacaggtcagcaggttagaccggcttttgaccatattcatcgctatttaaaacctgtggcaaaatgaacgcttattttacagactttctaatttgaccagatttcttaatgaatagacacagaattaactaaaaacagtctcaccccatgtagtgcgccgtgtcctgagagaggtgccctccctacgaggagggaagaacaggccctggggtgcagaggcccggcacgtagagaacccagatagacgccggtggtggaactggtcaaactccacgcccgcctgggaggttgtcaggttgctgtggatgtaaggataggaggtgcccagtgctccgctcaaggaaggctggatctgggccccacctacagagagggctcagggctggaccgggggcattgtgtgcttgggccgacccgggccggtggcagacgctgttctctgtcgggagatttgcgtccccaggaccctgttacacagtgggctgttgggttggtggctggcttttcctctatggacttcctcttcctgccccacctgcataggcacacacaccttgaatctgcaccctctggagggcatctgtactcctgtgcaaaatgcccagtccagagacaaaacctcagactttgtgcacctaggtttccttctcagcagcggagactgttctttgagttgccttgaagtggaggccgagcggctgcgggcccttcgcctccctgcggctgaccttgatgtagctttaagtcacactagactgcagaggggtccgaggccagaaacccctgtcctgcatcagactttcattcccacgttcttaggctttgttactgatacctcaaatcggaagttttagttctgagaaaggcaagtcagcgttcttgaaatgcctgactggtagatatgcaactctggcctccagtcttccatgaaaataaatgctgcctggacccccacccagaccacacactgacggccggctccggcggtgcccacccctcaggctggcccggcacccaagactggccacagccagctctgtcagcatgttgtgctcggacaagctgtttccttcttctgaccaacccaggtgtgacctggggatgcagagctttctgttttgggtgttgggagaagcagcaggaaggagtcgccagatgatcaagctccccctttgctgtcatctgtgaatgagcttcgccaggtggtgggcacctgggagccatgcagaggctgtggtgctgagttagactccaggtactttgtggtcaaaggaaatcgcctagctccaggctgtgttaggacagtattagcatgaaggctgtgcgaccatcatgcctgctgatccttgaggcaggcctggtccagaaaactctgggtcagtgactgcgcagggccagccgctaccaggacggccctgaaacaggacacatctgttttttgtccctcaccctgggcaggccgcgtcacaatcacagtcctcctcctccccaccctgacgtctgagcgcagggcttgaattgttagtcccaactctggccaaagatacttttttccagagacagaggccaggaggcagtgaggggagccccgcggggaggcggcggcgactgccacagcccttccagcctgtcttgctggccgccctggttcatatttgagtttaattgtactgaccctggacccagataagcagcaactttgtgtctttggggtcacagaacattttggggcagtttaatgtggtaccaaactgaaaataggagctatttatagatggagcagcacttagtgcttcatagaaagcaatgcctatttttaaagttacaaacgcagatatctacatagatatgctttgctgagaagttaggtctgtggtagaccagaaaccacaaattgactttttttcttagaaaatatttctatttgcggtaaatatagtaatatgtaaataatgtacatctgttgatttctggagtgtctgttattcaatgatgtatatactcccacagctcgcatgaaggaacagcctctattgatacttggttgtaaagtgaagtaagattggagggtggatggctgtcagagctcttgcagatactgtgttcactaaataaaaatcacatgtattgttaaaaaaaaaaaaaaaaa

SEQ ID NO: 3 - protein, 399 aa, Homo sapiens
MEDGVYEPPDLTPEERMELENIRRRKQELLVEIQRLREELSEAMSEVEGLEANEGSKTLQRNRKMAMGRKKFNMDPKKGIQFLVENELLQNTPEEIARFLYKGEGLNKTAIGDYLGEREELNLAVLHAFVDLHEFTDLNLVQALRQFLWSFRLPGEAQKIDRMMEAFAQRYCLCNPGVFQSTDTCYVLSFAVIMLNTSLHNPNVRDKPGLERFVAMNRGINEGGDLPEELLRNLYDSIRNEPFKIPEDDGNDLTHTFFNPDREGWLLKLGGRVKTWKRRWFILTDNCLYYFEYTTDKEPRGIIPQENLSIREVDDPRKPNCFELYIPNNKGQLIKACKTEADGRVVEGNHMVYRISAPTQEEKDEWIKSIQAAVSVDPFYEMLAARKKRISVKKKQEQP

SEQ ID NO: 4 - protein, 1602 aa, Homo sapiens
GGAAGAGTCTTTTCAGCGCTGAGGACTGGCGCTGAGGAGGCGGCGGTGGCTCCCGGGGCGTTTGAGCGGGCTCACCCGAGCCCGCGGGCCAACGCGGATCCAGGCCCGACTGGCGGGACCGCCCCGGATTCCCCGCGGGCCTTCCTAGCCGCCATGGAGGACGGCGTCTATGAACCCCCAGACCTGACTCCGGAGGAGCGGATGGAGCTGGAGAACATCCGGCGGCGGAAGCAGGAGCTGCTGGTGGAGATTCAGCGCCTGCGGGAGGAGCTCAGTGAAGCCATGAGCGAGGTGGAGGGGCTGGAGGCCAATGAGGGCAGTAAGACCTTGCAACGGAACCGGAAGATGGCAATGGGCAGGAAGAAGTTCAACATGGACCCCAAGAAGGGGATCCAGTTCTTGGTGGAGAATGAACTGCTGCAGAACACACCCGAGGAGATCGCCCGCTTCCTGTACAAGGGCGAGGGGCTGAACAAGACAGCCATCGGGGACTACCTGGGGGAGAGGGAAGAACTGAACCTGGCAGTGCTCCATGCTTTTGTGGATCTGCATGAGTTCACCGACCTCAATCTGGTGCAGGCCCTCAGGCAGTTTCTATGGAGCTTTCGCCTACCCGGAGAGGCCCAGAAAATTGACCGGATGATGGAGGCCTTCGCCCAGCGATACTGCCTGTGCAACCCTGGGGTTTTCCAGTCCACAGACACGTGCTATGTGCTGTCCTTCGCCGTCATCATGCTCAACACCAGTCTCCACAATCCCAATGTCCGGGACAAGCCGGGCCTGGAGCGCTTTGTGGCCATGAACCGGGGCATCAACGAGGGCGGGGACCTGCCTGAGGAGCTGCTCAGGAACCTGTACGACAGCATCCGAAATGAGCCCTTCAAGATTCCTGAGGATGACGGGAATGACCTGACCCACACCTTCTTCAACCCGGACCGGGAGGGCTGGCTCCTGAAGCTGGGGGGCCGGGTGAAGACGTGGAAGCGGCGCTGGTTTATCCTCACAGACAACTGCCTCTACTACTTTGAGTACACCACGGACAAGGAGCCCCGAGGAATCATCCCCCAGGAGAATCTGAGCATCCGAGAGGTGGACGACCCCCGGAAACCGAACTGCTTTGAACTTTACATCCCCAACAACAAGGGGCAGCTCATCAAAGCCTGCAAAACTGAGGCGGACGGCCGAGTGGTGGAGGGAAACCACATGGTGTACCGGATCTCGGCCCCCACGCAGGAGGAGAAGGACGAGTGGATCAAGTCCATCCAGGCGGCTGTGAGTGTGGACCCCTTCTATGAGATGCTGGCAGCGAGAAAGAAGCGGATTTCAGTCAAGAAGAAGCAGGAGCAGCCCTGACCCCCTGCCCCCAACTCCATTATTTATTACGGAGCTGCCCCGCCTGGGTGGCCGGACCCCTGGGCCTTGGGGCTGTGGATCCTGGTTCCCTGTTTGGAAAATTCACCACCTCTAGCTCCTCACTGTTCTTTGTAATTAACACGCTGTTGGTAATCTTATTAATTATTTAACCACTTGGAAAAAAAAAAATFFNPDREGWLLKLGGRVKTWKRRWFILTDNCLYYFEYTTDKEPRGIIPLENLSIREVED

SEQ ID NO: 5 - protein, 121 aa, Homo sapiens
PDREGWLLKLGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVSVDPFY